(19) United States
(12) Patent Application Publication
(10) Pub. No.: US 2019/0336472 A1
(43) Pub. Date: Nov. 7, 2019

(54) MEDICINAL COMPOUNDS AND NUTRITIONAL SUPPLEMENTS

(71) Applicant: RECEPTOR HOLDINGS, INC., Seattle, WA (US)

(72) Inventors: Andrea Leone-Bay, Ridgefield, CT (US); Gregory Wesner, Bainbridge Island, WA (US)

(73) Assignee: RECEPTOR HOLDINGS, INC., Seattle, WA (US)

(21) Appl. No.: 16/474,480

(22) PCT Filed: Jan. 3, 2018
(86) PCT No.: PCT/US18/12261

§ 371 Date: Jun. 27, 2019

Related U.S. Application Data
(60) Provisional application No. 62/441,908, filed on Jan. 3, 2017.

(51) Int. Cl. A61K 31/352; A61K 31/192; A61K 9/00; A23L 33/105

(52) U.S. Cl. CPC A61K 31/352; A23L 33/105; A61K 9/0053; A61K 31/192

(57) ABSTRACT

Synthetic cannabinoid medicinal compounds or nutritional supplements in various carrier combinations are described. The carriers can include N-acylated fatty amino acids, penetration enhancers, and/or various other beneficial carriers. The synthetic cannabinoid composition/carrier combinations can create administration benefits.

### CROSS REFERENCE TO RELATED APPLICATION

This application claims priority to U.S. Provisional Patent Application No. 62/441,908 filed on Jan. 3, 2017, which is incorporated herein by reference in its entirety as if fully set forth herein.

### FIELD OF THE DISCLOSURE

The current disclosure provides synthetic cannabinoids in various carrier combinations. The carriers can include N-acylated fatty amino acids, penetration enhancers, and/or various other beneficial carriers. The compositions including synthetic cannabinoid and carrier can create administration benefits.

### BACKGROUND OF THE DISCLOSURE

Cannabinoids are a diverse class of compounds that interact with and activate cannabinoid receptors. There are three classes of cannabinoids: 1) endocannabinoids, which are naturally produced in the body by humans and other animals, 2) phytocannabinoids, which are produced by plants, and 3) synthetic cannabinoids, which are chemically produced cannabinoids. Synthetic cannabinoids can be identical to cannabinoids that are found in nature, or can be compounds that do not exist in nature.

Endocannabinoids are part of the endocannabinoid system, which refers to endogenous cannabinoids and cannabinoids receptors. Cannabinoid receptors are expressed in various cell types, including brain cells and immune cells. An example of an endocannabinoid is anandamide, which is a fatty acid neurotransmitter that interacts with cannabinoid receptors and is involved in regulating the sensations of hunger, motivation, and pleasure.

Phytocannabinoids are produced by various types of plants, but the most well-known cannabinoid-producing plant is Cannabis. Cannabis sativa is an example of a plant in the Cannabis genus. Other plants of the Cannabis genus include Cannabis indica and Cannabis ruderalis. Hybrids between Cannabis sativa and Cannabis indica are common.

Cannabis plants produce over 100 cannabinoids, many of which have known therapeutic potential. The most well-known cannabinoids produced by Cannabis include (−)-trans-delta-9-tetrahydrocannabinol (THC, Δ⁹-THC), cannabidiol (CBD) and cannabinol. Other cannabinoids found in the Cannabis plant include cannabigerol, cannabidivarin, tetrahydrocannabivarin, and cannabichromene.

Plants other than Cannabis are known to produce cannabinoids. Examples of such plants include Echinacea purpurea, Echinacea angustifolia, Acmella oleracea, Helichrysum umbraculigerum, and Radula marginata. Cannabinoids isolated from Echinacea include lipophilic alkamides (alkylamides). Over 25 different alkylamides have been identified. They include the cis/trans isomers dodeca-2E,4E,8Z,10E/Z-tetraenoic-acid isobutylamide.

THC, the primary psychoactive compound of Cannabis, is the most extensively studied cannabinoid and has many well-established health benefits. THC is prescribed under the pharmaceutical drug name dronabinol, and is FDA approved for use as an appetite stimulant for HIV- and AIDS-related weight loss and for chemotherapy-induced nausea and vomiting. Many other medical uses of THC are being investigated, and research indicates that THC may have anti-tumor activity (Guzman M, Nat Rev Cancer. 2003. 3:745-55), anti-inflammatory effects (Gaiffal E, et al. Allergy. 2013. 68(8): 994-1000), and analgesic effects (Pharm. J. 259, 104, 1997 and in Pharm. Sci. 3, 546, 1997).

THC exhibits complex effects on the central nervous system (CNS), including central sympathomimetic activity. THC demonstrates effects on mood, cognition, memory, appetite and perception. These effects appear to be dose related. After oral administration, THC has an onset of action of 0.5 to 1 hour and a peak effect at 2-4 hours. The duration of action for psychoactive effects is 4-6 hours, but the appetite stimulant effect may continue for 24 hours or longer after administration. THC is almost completely absorbed (90-95%) after single oral doses. However, due to a combined effect of first pass hepatic metabolism and high lipid solubility only 10-20% of the administered dose reaches the systemic circulation.

Nabilone, a synthetic cannabinoid not found in nature, is another cannabinoid that has numerous medical uses. Nabilone, which is structurally very similar to THC, has been reported to be an anti-emetic and anxiolytic, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7).

Another cannabinoid with well-documented health benefits is CBD. In contrast to THC, CBD does not exert psychoactive effects. CBD is reported to have antidepressant (Zanelati T, et al. Journal of Pharmacology. 2010. 159(1):122-8;), anti-anxiety (Resstel B M, et al. Br J Pharmacol. 2009. 156(1):181-188), anti-inflammatory (Vuolo F, et al. Mediators of Inflammation. 2015. 538670), and neuroprotective effects (Campos A C, et al. Pharmacol Res. 2016. 112:119-127).

Compositions that include CBD and THC can also have health benefits. Nabiximols is a cannabis extract that contains a one-to-one ratio of CDB:THC and is commercially available as Sativex® (GW Pharmaceuticals plc, Wilshire, United Kingdom). Nabiximols is used to treat spasticity (muscle spasms and stiffness) in multiple sclerosis patients.

Additional uses for cannabinoids include treatment of addiction (De Vries, et al., Psychopharmacology (Berl). 2003 July; 168(1-2):164-9); ADHD (O'Connell and Che, Harm Reduction Journal. 2007; 4:16); alcoholism (Basavarajappa & Hungund, Alcohol. 2005 January-February; 40(1):15-24); Alzheimer's disease (Eubanks et al., Mol Pharm. 2006 November-December; 3(6):773-7); amyotrophic lateral sclerosis (ALS) (Raman et al., Amyotroph Lateral Scler Other Motor Neuron Disord. 2004 March; 5(1):33-9); anxiety (The British Journal of Psychiatry February 2001, 178 (2) 107-115); asthma (Tashkin et al., American Review of Respiratory Disease, 1975; 112, 377); auto-immune diseases (Lyman et al., J Neuroimmunol. 1989 June; 23(1):73-81); bacterial infections (Nissen et al., Fitoterapia. 2010 July; 81(5):413-9); bone loss (Bab et al., Ann Med. 2009; 41(8):560-7); brain injury/stroke (Shohami et al., Br J Pharmacol. 2011 August; 163(7):1402-10); cancer (Guindon & Hohmann, Br J Pharmacol. 2011 August; 163(7):1447-63); heart disease (Walsh et al., Br J Pharmacol. 2010 July; 160(5):1234-42); Huntington's disease (Lastres-Becker et al., J Neurochem. 2003 March; 84(5):1097-109); inflammation (AAPS J. 2009 March; 11(1): 109-119); Parkinson's disease (Sieradzan et al., Neurology. 2001 Dec. 11; 57(11):2108-11); and psoriasis (Trends Pharmacol Sci. 2009 August; 30(8): 411-420).

Additional documented uses for the cannabinoids include treating acquired hypothyroidism, acute gastritis, agoraphobia, ankyloses, arthritis, Asperger's syndrome, atherosclerosis, autism, bipolar disorder, blood disorders, cachexia, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, hepatitis, herpes, hypertension, impotence, incontinence, infant mortality, inflammatory bowel disease (IBD), insomnia, liver fibrosis, mad cow disease, menopause, migraine headaches, motion sickness, MRSA, muscular dystrophy, nail patella syndrome, neuroinflammation, nicotine addiction, obesity, obsessive compulsive disorder (OCD), pancreatitis, panic disorder, periodontal disease, phantom limb pain, poison ivy allergy, premenstrual syndrome (PMS), proximal myotonic myopathy, post-traumatic stress disorder (PTSD), Raynaud's disease, restless leg syndrome, schizophrenia, scleroderma, septic shock, shingles herpes zoster), sickle cell disease, seizures, sleep apnea, sleep disorders, stress, stuttering, temporomandibular joint disorder (TMJ), tension headaches, tinnitus, Tourette's syndrome, traumatic memories, wasting syndrome, and withdrawal syndrome.

### SUMMARY OF THE DISCLOSURE

The current disclosure provides compositions including synthetic medicinal compounds or nutritional supplements in various carrier combinations.

In particular embodiments, the compositions include one or more synthetic cannabinoids and one or more carriers.

In particular embodiments, the carriers include N-acylated fatty amino acids, absorption enhancing agents, and/or various other beneficial carriers, such as surfactants, detergents, azones, pyrrolidones, glycols and bile salts. In particular embodiments, N-acylated fatty amino acids can be linear, branched, cyclic, bicyclic, or aromatic including, for example, 1-50 carbon atoms.

The compositions, provided herein, can create various administration benefits in providing therapeutically or nutritionally effective amounts in a variety of conditions. Exemplary administration benefits include increased absorption, increased bioavailability, faster onset of action, higher peak concentrations, faster time to peak concentrations, increased subjective therapeutic efficacy, increased objective therapeutic efficacy, improved taste, and improved mouthfeel. In particular embodiments, one or more administration benefits can increase compliance with a dose schedule.

### BRIEF DESCRIPTION OF THE FIGURES

FIGS. 1A and 1B show an established correlation between water-solubility and the ability of SNAC to improve a molecule's absorption. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each and the extent to which SNAC improves absorption. As the water solubility of the molecule increases, SNAC's ability to enhance its absorption also increases. FIG. 1B plots the aqueous solubility of heparin, acyclovir, rhGH, PTH, MT-II, GLP-1, calcitonin, yy peptide, and THC according to the logarithmic trendline derived from FIG. 1A.

FIG. 2 provides exemplary structures of cannabinoids that can be synthetically derived (THC, nabilone, CBD, 7-OH-CBD, CBDV, 7-OHCBDV, and formulas I-XVI).

FIG. 3 provides modified amino acids of compounds I-XXXV.

FIG. 4 provides fatty acid amino acids of Formulas (a), (b), (c), (d), (e), (f), (g), (h), (i), (j), (k), (l), (m), (n), (o), (p), (q), (r) and (s) wherein R1 is an alkyl group including 5 to 19 carbon atoms, R2 is H (i.e. hydrogen) or CH3 (i.e. methyl group), R3 is H, and R4 is an amino acid side chain or covalently attached to R2 via a (CH₂)₃ group; or a salt or the free acid form thereof.

FIGS. 5A and 5B provide the average results of the study comparing onset and duration of action of orally administered cannabis/N-[8-(2-hydroxybenzoyl) amino] caprylate (SNAC) formulation and cannabis (without SNAC) formulation. FIG. 5A provides a bar graph of the results, with the SNAC formulation results depicted with black bars, and the results for the formulation without SNAC depicted with white bars. FIG. 5B provides a line graph of the results, with the SNAC formulation results depicted with circles, and the results for the formulation without SNAC depicted with triangles.

FIGS. 6A-6F provide the results for each individual participant in the study comparing onset and duration of action of orally administered cannabis/N-[8-(2-hydroxybenzoyl) amino] caprylate (SNAC) formulation (dark gray bars) and cannabis (without SNAC) formulation (light gray bars). FIG. 6A shows results for Study Participant No. 1 (“S1”); FIG. 6B shows results for Study Participant No. 2 (“S2”); FIG. 6C shows results for Study Participant No. 3 (“S3”); FIG. 6D shows results for Study Participant No. 4 (“S4”); FIG. 6E shows results for Study Participant No. 5 (“S5”) and FIG. 6F shows results for Study Participant No. 6 (“S6”).

FIG. 7 shows a comparison of intensity, duration and onset of action of orally administered cannabinoid formulations with a high SNAC dose (200 mg, “high dose”), a low SNAC dose (100 mg, “low dose”) and no SNAC (“control”).

FIG. 8 shows intensity, duration and onset of action of a cannabinoid formulated with SNAC administered orally (“PO”) compared to a cannabinoid administered by inhalation (“INH”).

FIG. 9 shows THC and CBD Cₘₐₓ and AUC following a single oral administration to rats.

FIG. 10 shows THC and CBD Cₘₐₓ (ng/ml) and AUC (hr*ng/mL) following a single oral administration to rats.

FIG. 11 shows intensity, duration and onset of action of orally administered cannabis/N-[8-(2-hydroxybenzoyl) amino] caprylic acid (NAC, “test”) formulation and cannabis only (without NAC, “control”) formulation.

### DETAILED DESCRIPTION

The current disclosure provides compositions including one or more synthetic cannabinoids in various carrier combinations. The compositions can be pharmaceutical and/or nutritional compositions. The nutritional compositions can be nutritional supplements. The synthetic cannabinoids in the compositions provide therapeutic benefits or nutritional benefits.

Synthetic cannabinoids include cannabinoids that are chemically produced. Synthetic cannabinoids also include cannabinoids that are found in nature, but are produced chemically. Synthetic cannabinoids also include chemically produced derivatives and analogs of cannabinoids.

The term “derivative” in chemistry refers to a compound that is obtained from a similar compound or a precursor compound by a chemical reaction.

The term “analog” (also “structural analog” or “chemical analog”) is used to refer to a compound that is structurally similar to another compound but differs with respect to a certain component, such as an atom, a functional group, or a substructure.

Examples of cannabinoids derived from plants include cannabigerol (CBG), cannabichromene (CBC), cannabidiol (CBD), cannabinol (CBN), tetrahydrocannabinol (THC), iso-THC, cannabielsoin (CBE), cannabicyclol (CBL), cannabidivarin (CBDV), tetrahydrocannabivarin (THCV), and cannabicitran (CBT). In particular embodiments, synthetic cannabinoids include natural cannabinoids that are synthesized chemically and also their analogs and derivatives. Derivatives of natural cannabinoids can include metabolites of cannabinoids which are disclosed in WO 2015/198078. For example, the metabolite of CBD includes 7-OH-CBD and the metabolite of CBDV includes 7-OH-CBDV.

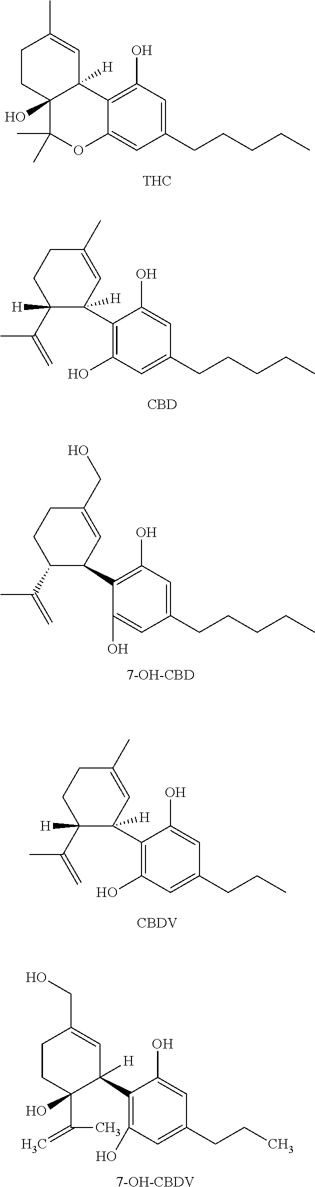

Other examples of synthetic cannabinoids include 3-carbamoyl-2-pyridone, and its derivatives and/or analogs disclosed in US 2008/0103139; pyrimidine derivatives and/or analogs disclosed in US 2006/0293354; carenadiol and its derivatives and/or analogs thereof disclosed in U.S. Pat. No. 4,758,597; cannabinoid carboxylic acids and their derivatives and/or analogs disclosed in WO 2013/045115; pyrido[3,2-E][1,2,4]triazolo[4,3-C]pyrimidine and its derivatives and/or analogs disclosed in WO 2008/118414; tetrahydro-pyrazolo[3,4-C] pyridine and its derivatives and/or analogs disclosed in WO 2007/112399; bicyclo[3.1.1]heptan-2-one cannabinoid and its derivatives and/or analogs disclosed in WO 2006/043260; resorcinol and its derivatives and/or analogs disclosed in WO 2005/0123051; dexanabinol compounds and their derivatives and/or analogs disclosed in WO 2004/050011; cannabimimetic lipid amide compounds and their derivatives and/or analogs disclosed in WO 2000/032200; nabilone and its derivatives and/or analogs disclosed in US 2010/0168066; 2-oxoquinolone compounds and their derivatives and/or analogs disclosed in US 2003/0191069; and 3,4-diaryl-4,5-dihydro-(h)-pyrazole-1-carboxamide and its derivatives and/or analogs disclosed in US 2011/0137040.

In particular embodiments, 3-carbamoyl-2-pyridone and its derivatives and/or analogs include methyl 3-methyl-2-{[2-oxo-1-(2-oxo-ethyl)-1,2,5,6,7,8,9,10-octahydro-cycloocta[b]pyridine-3-carbonyl]-amino}-butyrate; dimethyl 2-[(1-cyclohexylmethyl-5,6-dimethyl-2-oxo-1,2-dihydropyridine-3-carbonyl)-amino]-succinate; and methyl 2-{[1-(3-methoxycarbonyamino-propyl)-2-oxo-1,2,5,6,7,8,9,10-octahydro-cycloocta[b]pyridine-3-carbonyl]-amino}-2-methyl-propionate.

In particular embodiments, pyrimidine derivatives and/or analogs include a compound having Formula (I) (2-((2,4-dichlorophenyl)amino)-N-((tetrahydro-2H-pyran-4-yl)methyl)-4-(trifluoromethyl)pyrimidine-5-carboxamide),

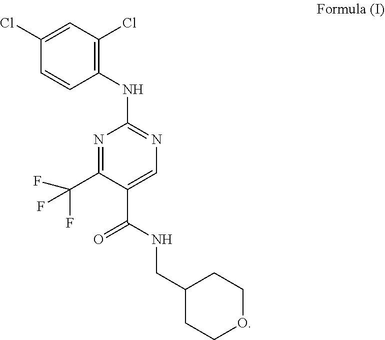 [SMILES: O=C(NCC1CCOCC1)c1cnc(Nc2ccc(Cl)cc2Cl)nc1C(F)(F)F]

Other pyrimidine derivatives and/or analogs include 2-(3-Chlorophenylamino)-4-trifluoromethylpyrimidine-5-carboxylic acid cyclohexylmethyl-amide; 2-Phenylamino-4-trifluoromethylpyrimidine-5-carboxylic acid cyclohexylmethyl-amide; 1-[2-(2,3-Dichlorophenylamino)-4-trifluoromethylpyrimidin-5-yl]-1-morphol-in-4-yl-methanone; 1-[2-(2,4-Dichlorophenylamino)-4-trifluoromethylpyrimidin-5-yl]-1-morphol-in-4-yl-methanone; and 2-(3-Chlorophenylamino)-4-trifluoromethylpyrimidin-5-carboxylic acid cyclopentylamide.

In particular embodiments, carenadiol and its derivatives and/or analogs include compounds having Formula (II),

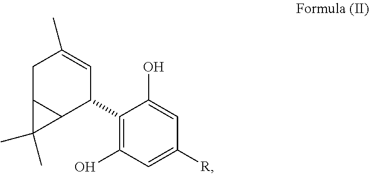

wherein R is a lower alkyl having 1 to 9 carbon atoms including isomeric forms such as i-butyl, n-butyl, and t-butyl. In particular embodiments, R is C₅H₁₁ or 1,1-dimethylheptyl.

In particular embodiments, cannabinoid carboxylic acids and their derivatives and/or analogs include compounds having Formula (III), (IV), (V), or (VI),

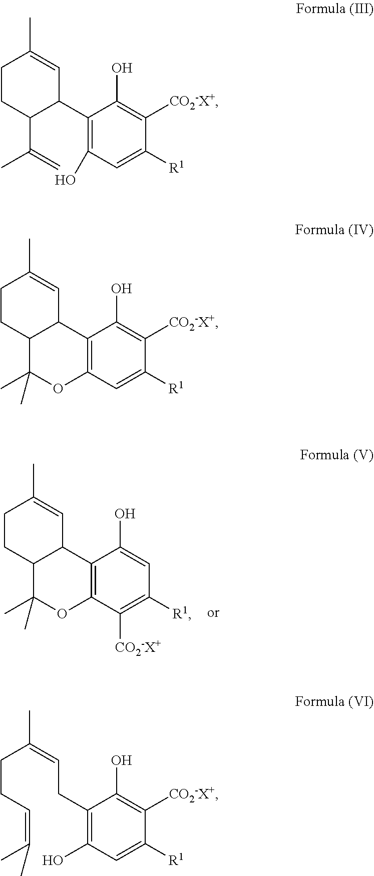

wherein:
R¹ is a straight-chain, branched or cyclic hydrocarbon residue with one C atom to 12 C atoms; and
X⁺ is NH₄⁺, mono-, di- or trivalent metal ions; or primary, secondary, tertiary or quaternary organic ammonium ions with up to 48 C atoms, which may bear still further functional groups.

Examples of multivalent ammonium ions include N,N-dicyclo-hexylamine-H⁺ and N,N-dicyclohexyl-N-ethylamine-H⁺. X⁺ can also be the hydrogen cation of a pharmaceutical active substance with at least one basic nitrogen atom, such as for example morphine, methadone (or an enantiomer thereof) or hydromorphone.

In particular embodiments, pyrido[3,2-E][1,2,4]triazolo[4,3-C]pyrimidine and its derivatives and/or analogs include 5-tert-butyl-8-(2-chlorophenyl)-9-(4-chlorophenyl)pyrido[3,2-e][1,2,4]triazolo[4,3-c]pyrimidin-3(2H)-one; 8-(4-bromo-2-chlorophenyl)-5-tert-butyl-9-(4-chlorophenyl)pyrido[3,2-e][1,2,4]triazolo[4,3-c]pyrimidin-3(2H)-one; 5-tert-butyl-9-(4-chlorophenyl)-8-(2-methylphenyl)pyrido[3,2-e][1,2,4]triazolo[4,3-c]pyrimidin-3(2H)-one; 9-(4-bromophenyl)-5-tert-butyl-8-(2-chlorophenyl)pyrido[3,2-e][1,2,4]triazolo[4,3-c]pyrimidin-3(2H)-one; and 5-tert-butyl-8-(2-chlorophenyl)-9-(4-chlorophenyl)pyrido[3,2-e][1,2,4]triazolo[4,3-c]pyrimidine.

In particular embodiments, tetrahydro-pyrazolo[3,4-C] pyridine and its analogs and/or derivatives include compounds having Formula (VII), (VIII), (IX), (X), or (XI),

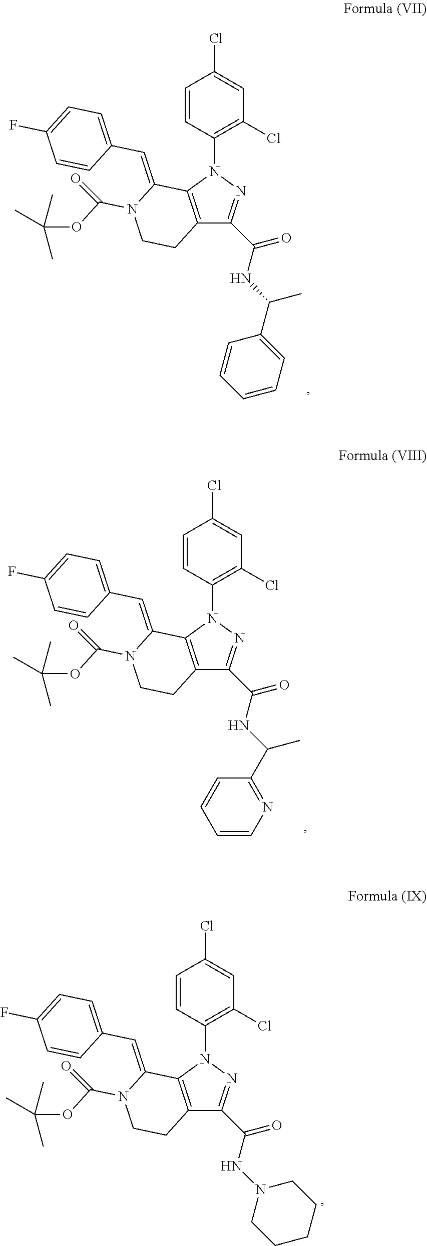 [SMILES: C[C@@H](NC(=O)c1nn(-c2ccc(Cl)cc2Cl)c2c1CCN(C(=O)OC(C)(C)C)/C2=C\c1ccc(F)cc1)c1ccccc1 ; CC(NC(=O)c1nn(-c2ccc(Cl)cc2Cl)c2c1CCN(C(=O)OC(C)(C)C)/C2=C\c1ccc(F)cc1)c1ccccn1 ; CC(C)(C)OC(=O)N1CCc2c(C(=O)NN3CCCCC3)nn(-c3ccc(Cl)cc3Cl)c2/C1=C/c1ccc(F)cc1]

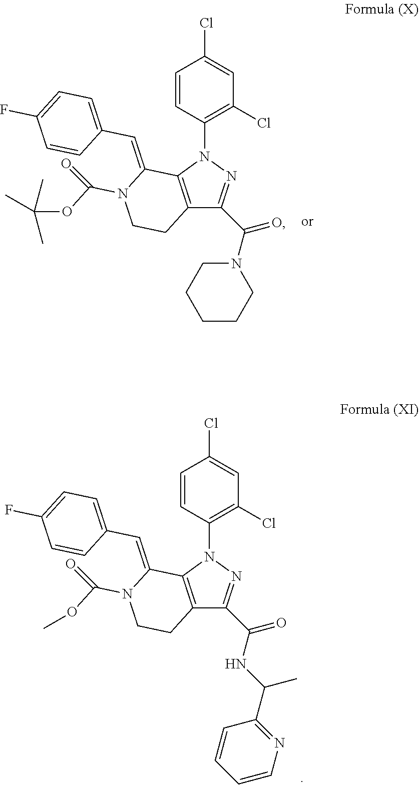 [SMILES: CC(C)(C)OC(=O)N1CCc2c(C(=O)N3CCCCC3)nn(-c3ccc(Cl)cc3Cl)c2/C1=C/c1ccc(F)cc1 ; "or" ; COC(=O)N1CCc2c(C(=O)NC(C)c3ccccn3)nn(-c3ccc(Cl)cc3Cl)c2/C1=C/c1ccc(F)cc1]

In particular embodiments, bicyclo[3.1.1]heptan-2-one cannabinoids and their derivatives and/or analogs include compounds having Formula (XII),

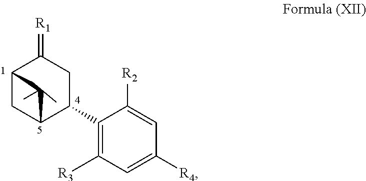

having a specific stereochemistry wherein C-4 is S, the protons at C-1 and C-5 are cis in relation to one another and the protons at C-4 and C-5 are trans; and wherein:
R₁ is (a) 0 or S; (b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR″, and C₁-C₆ alkyl-N(R′″)₂, wherein at each occurrence R″ is independently selected from the group consisting of hydrogen or saturated or unsaturated, linear, branched or cyclic C₁-C₁₂alkyl; or (c) NR″ or N—OR″ wherein R″ is as previously defined;
R₂ and R₃ are each independently (a) —R″, —OR″, —N(R″)₂, —SR″, —S(O)(O)NR″, wherein at each occurrence R″ is as previously defined; (b) —S(O)R^{b}, —S(O)(O)R^{b} wherein R^{b} is selected from the group consisting of hydrogen, saturated or unsaturated, linear or branched C₁-C₆alkyl, C₁-C₆alkyl-OR″, and C₁-C₆alkyl-N(R″)₂, wherein R″ is as previously defined; or (c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is (a) R wherein R is selected from the group consisting of hydrogen, halogen, OR″, OC(O)R′″, C(O)OR′″, C(O)R′″, OC(O)OR′″, CN, N(R′″)₂, NC(O)R′″, NC(O)OR′″, C(O)N(R′″)₂, NC(O)N(R′″)₂, and SR″, wherein at each occurrence R″ is as previously defined; (b) a saturated or unsaturated, linear, branched or cyclic C₁-C₁₂ alkyl-R wherein R is as previously defined; (c) an aromatic ring which can be further substituted at any position by R wherein R is as previously defined; or (d) a saturated or unsaturated, linear, branched or cyclic C₁-C₁₂ alkyl optionally terminated by an aromatic ring which can be further substituted as defined in (c).

In particular embodiments, resorcinol and its derivatives and/or analogs include compounds having Formula (XIII),

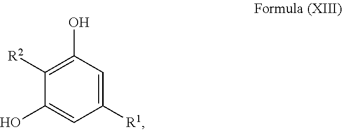

wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms.

In particular embodiments, resorcinol and its derivatives and/or analogs include compounds having Formula (XIII), wherein R¹ and R² are as follows:
R¹ is a straight alkyl chain of 5 to 8 carbon atoms, optionally substituted with one methyl group; and
R² is selected from geranyl optionally substituted with one —OH, and farnesyl optionally substituted with one —OH.

In particular embodiments, resorcinol and its derivatives and/or analogs include compounds having Formula (XIII), wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms; with the proviso that when R¹ is isononyl, R² is not geranyl.

In particular embodiments, resorcinol and its derivatives and/or analogs include compounds having Formula (XIII), wherein R¹ is (a) a straight or branched alkyl of 7 to 12 carbon atoms; (b) a group —O—R³, where R³ is a straight or branched alkyl of 5 to 9 carbon atoms, or a straight or branched alkyl substituted at the terminal carbon atom by a phenyl group; or (c) a group —(CH₂)ₙ—O-alkyl, where n is an integer from 1 to 7 and the alkyl group contains 1 to 5 carbon atoms.

In particular embodiments, resorcinol and its derivatives and/or analogs include compounds of Formula (XIII), wherein R² is a non-cyclic terpenoid carbon chain such as geranyl, farnesyl, and related non-cyclic terpenes and their isomers as well as other non-cyclic paraffinic or olefinic carbon chains.

In particular embodiments, resorcinol and its derivatives and/or analogs include compounds of Formula (XIII), wherein R¹ is dimethylheptyl and R² is geranyl.

In particular embodiments, dexanabinol compounds and their derivatives and/or analogs include high enantiomeric purity compounds having Formula (XIV),

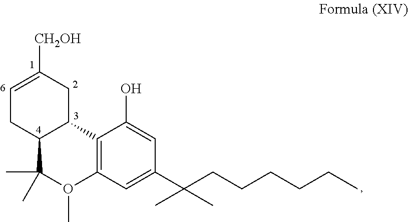

and having the (3S,4S) configuration and being in enantiomeric excess of at least 99.90% over the (3R,4R) enantiomer.

In particular embodiments, cannabimimetic lipid amide compounds and their derivatives and/or analogs include compounds having Formula (XV),

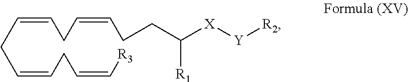

wherein:
X is one of the group consisting of C═O and NH, and Y is the other of that group. Expressed another way, X may be C═O and Y may be NH, or Y may be C═O and X may be NH, but both X and Y may not be the same group.
R₁ is H or an alkyl group. In particular embodiments, R₁ is H, CH₃, or (CH₃)₂;
R₂ is an alkyl, a substituted alkyl, an alkenyl or an alkynyl group. In particular embodiments, R₂ is CH(R) CH₂Z, CH₂CH(R)Z, or CH(R)(CH₂)nCH₂Z; R being H, CH, CH₃, CHCH, CH₂CF₃, or (CH₃)₂, Z being H, halogen, N₃, NCS, or OH; and n being selected from the group consisting of 0, 1 and 2.
R₃ is an alkyl, a substituted alkyl, an aryl, an alkylaryl, an O-alkyl, an O-alkylaryl, a cyclic and a heterocyclic group. O-alkyl and O-alkylaryl refer to groups in which an oxygen atom is interposed between carbon atoms on the anandamide portion and substituent group. Examples of such R₃ groups include cyclohexyl, cyclopentyl, alkylcyclohexyl, alkylcyclopentyl, piperidinyl, morpholinyi and pyridinyl. In particular embodiments, R₃ is n-C₅H₁₀Z′, n-C₆H₁₂Z′, n-C₇H₁₄Z′, or 1′,1′-C(CH₃)₂(CH₂)₅CH₂Z′; Z′ being H, halogens, CN, N₃, NCS, or OH.

In particular embodiments, cannabimimetic lipid amide compounds and their derivatives and/or analogs include compounds having Formula (XVI),

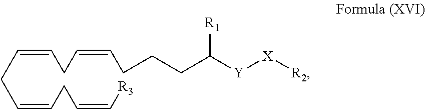

wherein:
Y is one of the group consisting of C═O and NH and X is the other of that group.
R₁ is H or an alkyl group. In particular embodiments, R₁ is H, CH₃, or (CH₃)₂.
R₂ is an alkyl, a substituted alkyl, an alkenyl, an alkynyl, an O-alkyl, a cyclic, a polycyclic, or a heterocyclic group. In particular embodiments, R₂ is

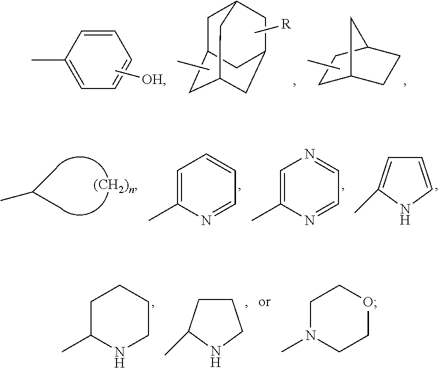

CH═CH₂, CH═C(CH₃)₂, C≡CH, CH₂OCH₃, CH(R)(CH₂)nCH₂Z, or CH₂CH(R)(CH₂)nZ; R being H, CH₃ or (CH₃)₂, Z being H, halogens, N₃, NCS, OH, or OAc; and n 0, 1, or 2; and
R₃ is an alkyl, a substituted alkyl, an aryl, an alkylaryl, an O-alkyl, an O-alkylaryl, a cyclic, or a heterocyclic group. In particular embodiments R₃ includes cyclohexyl, cyclopentyl, alkylcyclohexyl, alkylcyclopentyl, piperidinyl, morpholinyi and pyridinyl. In particular embodiments, R₃ is n-C₅H₁₀Z′, n-C₆H₁₂Z′, n-C₇H₁₄Z′, or 1′,1′-C(CH₃)₂CH₂)₅CH₂Z′; Z′ being H, halogen, CN, N₃, NCS, or OH.

In particular embodiments, nabilone and its derivatives and/or analogs include compounds having Formula (XVII):

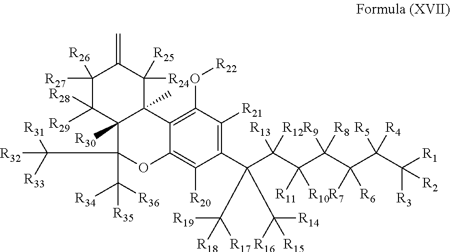

wherein:

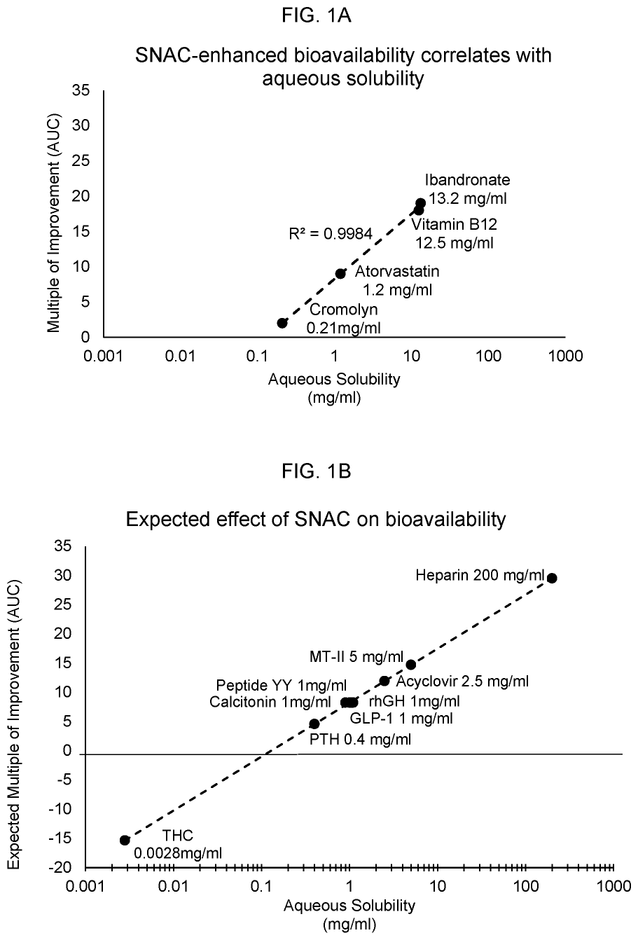

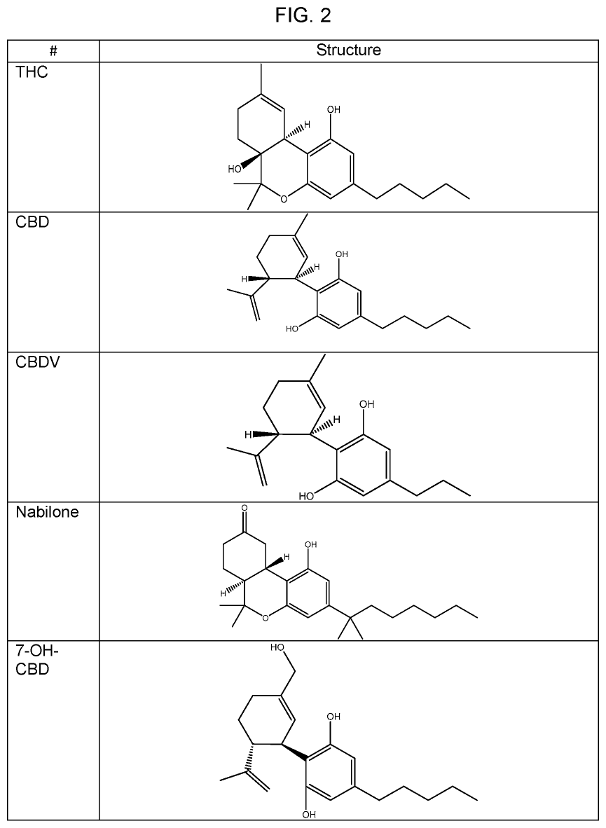

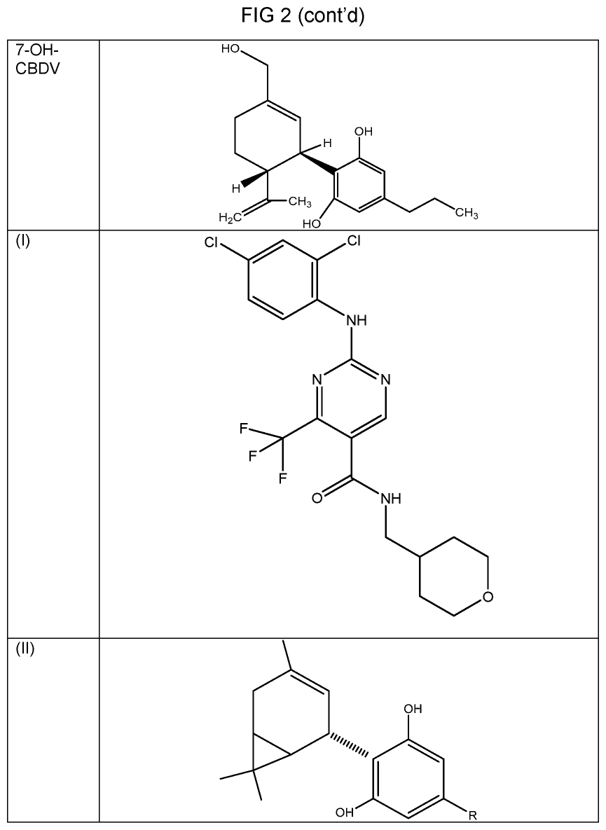

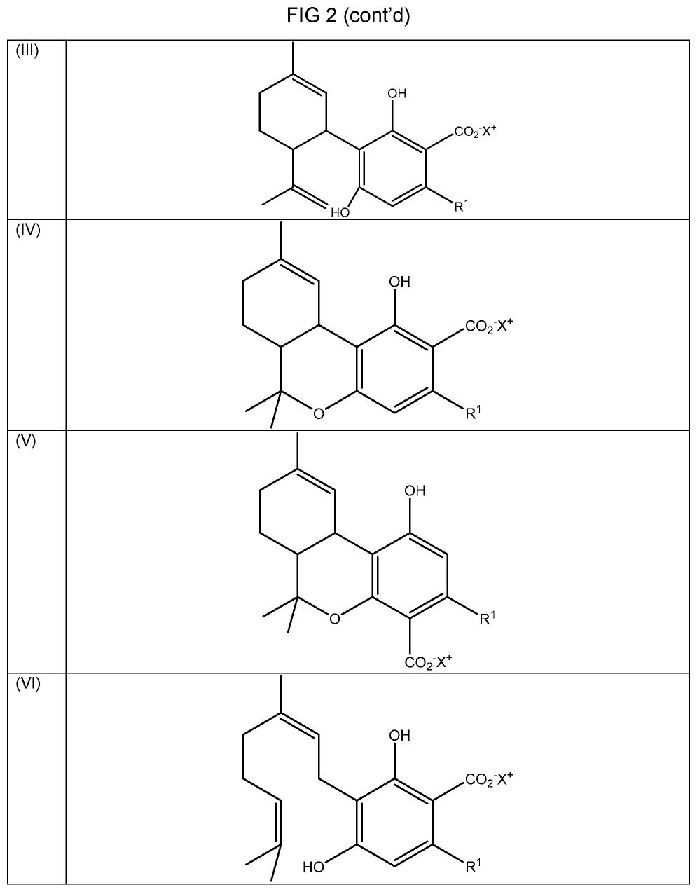

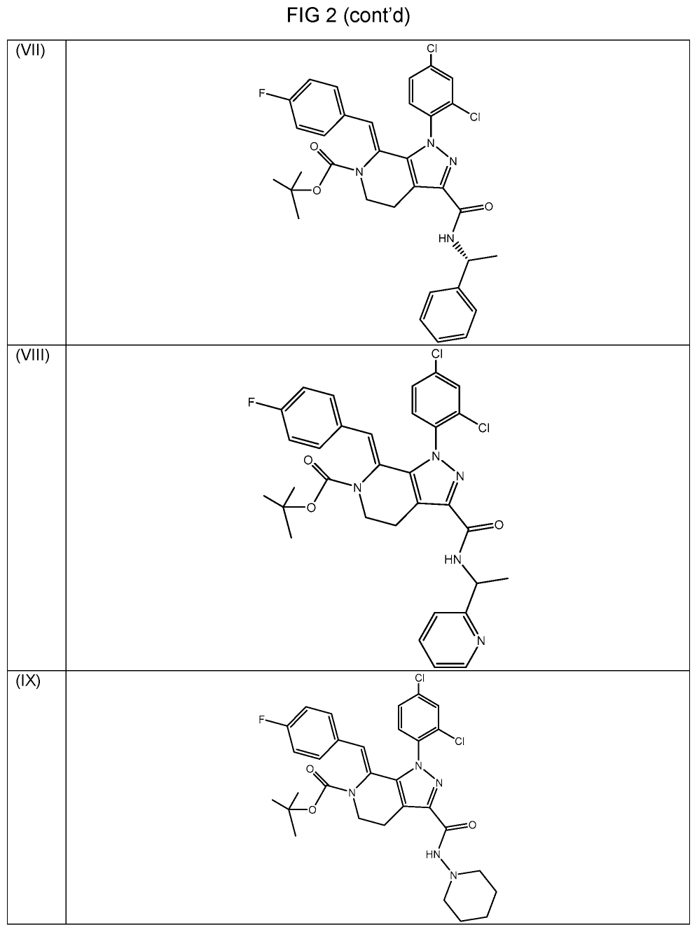

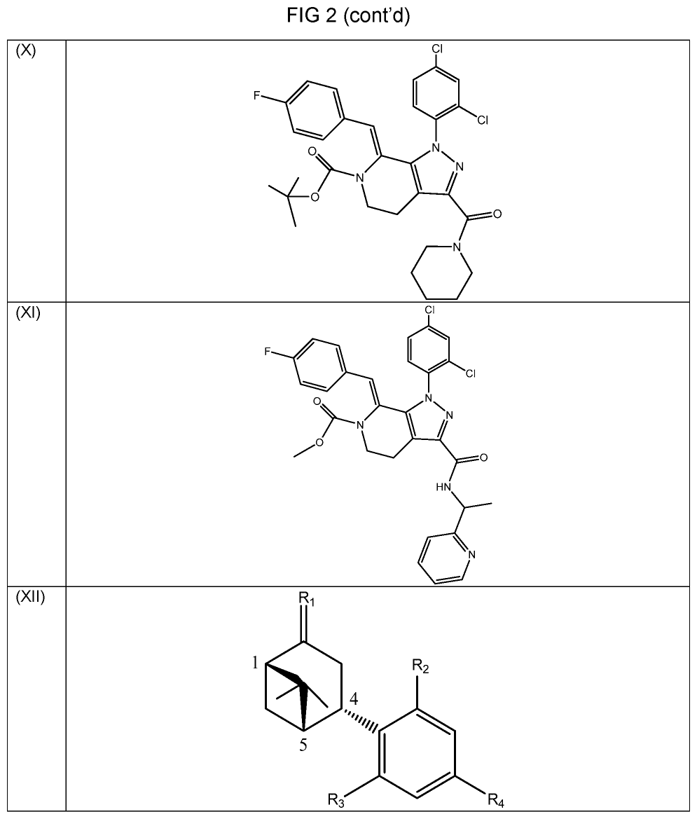

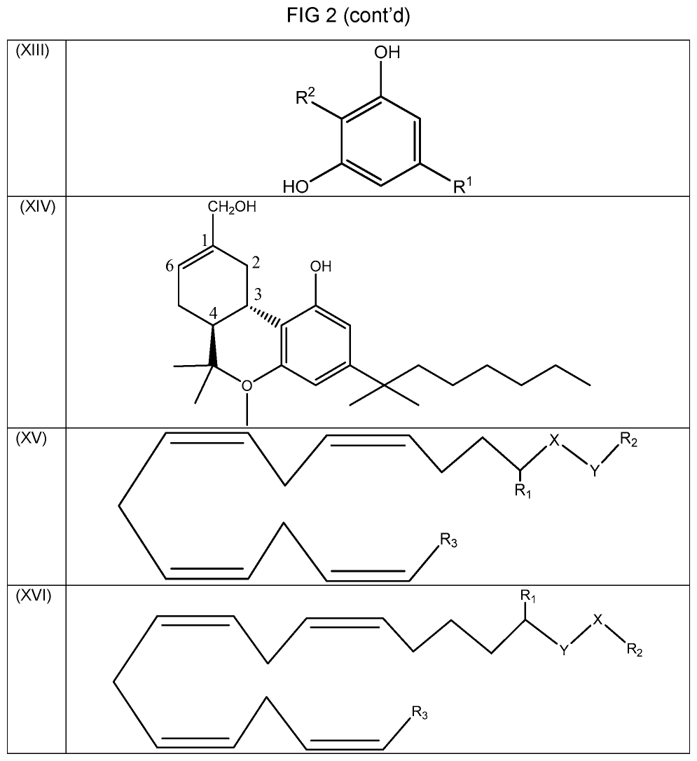

R¹-R³⁶ are independently selected from the group consisting of hydrogen and deuterium. Nabilone derivatives and/or analogs can refer to compounds wherein at least one of R¹-R³⁶ includes deuterium. For the chemical structure of nabilone, see FIG. 1.

Synthetic cannabinoids can be medicinal compounds and/or can be provided in combination with nutritional supplements. Synthetic cannabinoids are provided in therapeutically-effective amounts to treat a condition, such as those described in the Background of the Disclosure. Synthetic cannabinoids in combination with nutritional supplements claim a benefit related to a classical nutrient deficiency disease; describes how the supplement is intended to affect the structure or function of the human body; characterizes a documented mechanism by which the supplement acts to maintain such structure or function; and/or describes general well-being associated with consumption of the product. In particular embodiments, a nutritional supplement may not claim to diagnose, mitigate, treat, cure, or prevent a specific disease or class of diseases.

In particular embodiments, the compositions disclosed herein include carriers such as modified amino acids, a surfactant, a detergent, an azone, a pyrrolidone, a glycol, or a bile salt. An amino acid is any carboxylic acid having at least one free amine group and includes naturally occurring, non-naturally occurring and synthetic amino acids. Poly amino acids are either peptides or two or more amino acids linked by a bond formed by other groups which can be linked, e.g. an ester, anhydride, or an anhydride linkage. Peptides are two or more amino acids joined by a peptide bond. Peptides can vary in length from dipeptides with two amino acids to poly peptides with several hundred amino acids. See Chambers Biological Dictionary, editor Peter M. B. Walker, Cambridge, England: Chambers Cambridge, 1989, page 215. Di-peptides, tri-peptides, tetra-peptides, and penta-peptides can also be used.

Carriers which are modified amino acids include acylated fatty acid amino acids (FA-aa) or a salt thereof, which are typically prepared by modifying the amino acid or an ester thereof by acylation or sulfonation. Acylated fatty acid amino acids include N-acylated FA-aa or an amino acid acylated at its alpha amino group with a fatty acid.

Exemplary N-acylated fatty amino acid salts include sodium N-[8-(2-hydroxybenzoyl) amino] caprylate (SNAC). Other names for SNAC include Sodium-N-salicyloyl-8-aminocaprylate, Monosodium 8-(N-salicyloylamino) octanoate, N-(salicyloyl)-8-aminooctanoic acid monosodium salt, monosodium N-{8-(2-hydroxybenzoyl)amino}octanoate, or sodium 8-[(2-hydroxybenzoyl)amino]octanoate. SNAC has the structure:

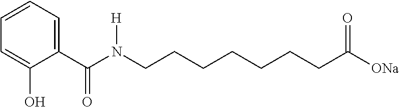

Salts of SNAC may also be used as a carrier.

Other forms of SNAC include:

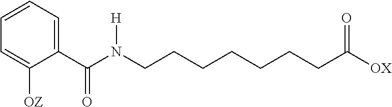

wherein X and Z are independently H, a monovalent cation, a divalent metal cation, or an organic cation. Examples of monovalent cations include sodium and potassium. Examples of divalent cations include calcium and magnesium. Examples of organic cations include ammonium and tetramethylammonium.

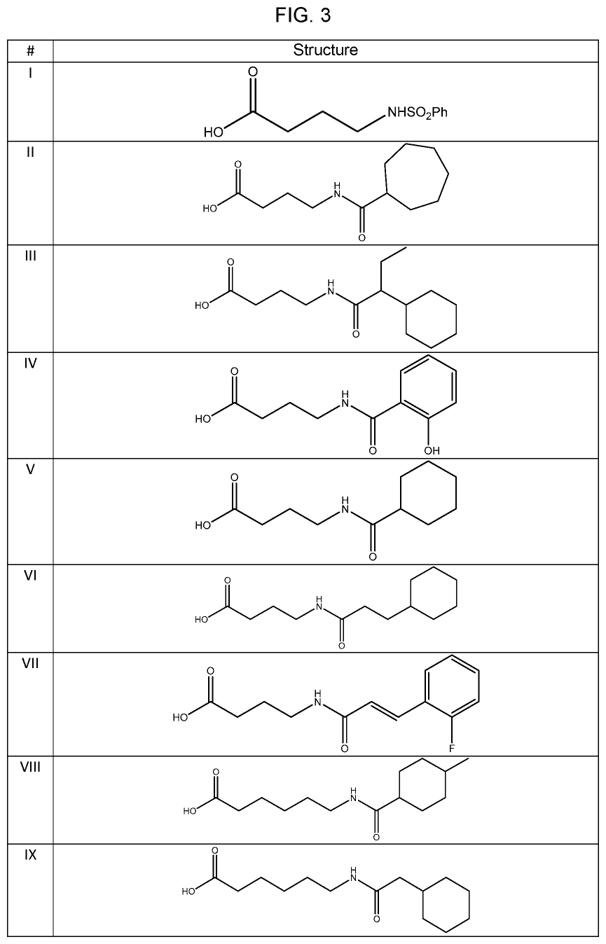

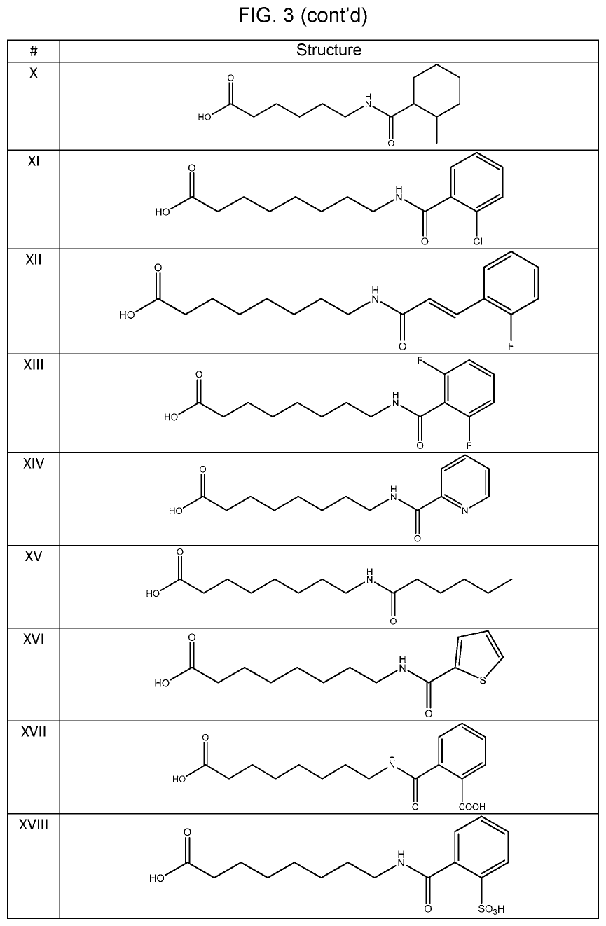

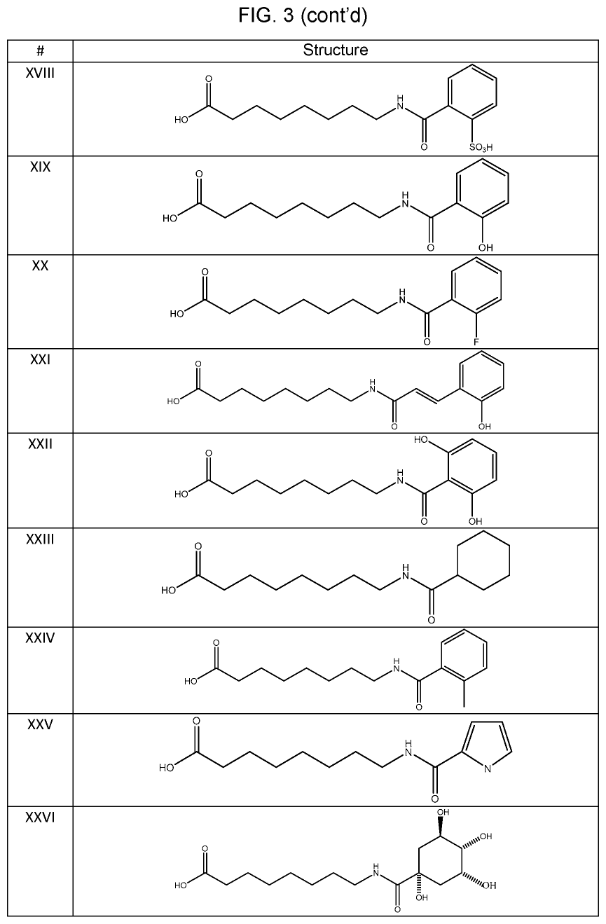

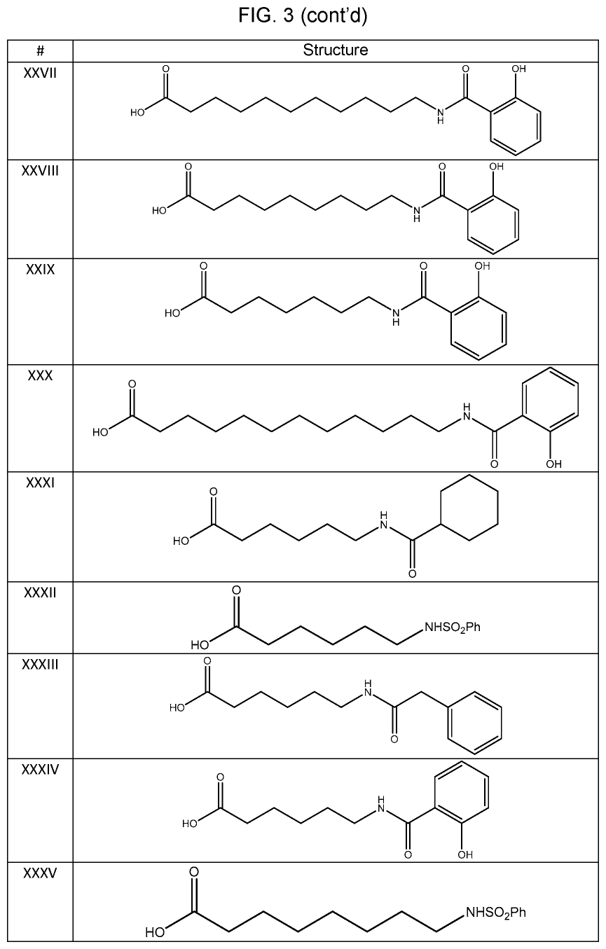

Exemplary modified amino acids, such as N-acylated FA-aas, are provided as compounds I-XXXV (see FIG. 3). Salts of these compounds and other N-acylated FA-aa can also be used as carriers.

Many of the compounds can be readily prepared from amino acids by methods within the skill of those in the art based upon the present disclosure. For example, compounds I-VII are derived from aminobutyric acid. Compounds VIII-X and XXXI-XXIIV are derived from aminocaproic acid. Compounds XI-XXVI and XXXV are derived from aminocaprylic acid. For example, the modified amino acid compounds above may be prepared by reacting the single amino acid with the appropriate modifying agent which reacts with free amino moiety present in the amino acids to form amides. Protecting groups may be used to avoid unwanted side reactions as would be known to those skilled in the art.

The amino acid can be dissolved in aqueous alkaline solution of a metal hydroxide, e.g., sodium or potassium hydroxide, and heated at a temperature ranging between 5° C. and 70° C., preferably between 10° C. and 40° C., for a period ranging between 1 hour and 4 hours, preferably 2.5 hours. The amount of alkali employed per equivalent of NH₂ groups in the amino acid generally ranges between 1.25 and 3 mmole, preferably between 1.5 and 2.25 mmole per equivalent of NH₂. The pH of the solution generally ranges between 8 and 13, preferably ranging between 10 and 12.

Thereafter, the appropriate amino add modifying agent is added to the amino add solution while stirring. The temperature of the mixture is maintained at a temperature generally ranging between 5° C. and 70° C., preferably between 10° C. and 40° C., for a period ranging between 1 and 4 hours. The amount of amino acid modifying agent employed in relation to the quantity of amino acid is based on the moles of total free NH₂ in the amino add. In general, the amino acid modifying agent is employed in an amount ranging between 0.5 and 2.5 mole equivalents, preferably between 0.75 and 1.25 equivalents, per molar equivalent of total NH₂ group in the amino acid.

The reaction is quenched by adjusting the pH of the mixture with a suitable acid, e.g., concentrated hydrochloric acid, until the pH reaches between 2 and 3. The mixture separates on standing at room temperature to form a transparent upper layer and a white or off-white precipitate. The upper layer is discarded, and the modified amino acid is collected from the lower layer by filtration or decantation. The crude modified amino acid is then dissolved in water at a pH ranging between 9 and 13, preferably between 11 and 13. Insoluble materials are removed by filtration and the filtrate is dried in vacuo. The yield of modified amino acid generally ranges between 30 and 60%, and usually 45%.

If desired, amino acid esters, such as, for example benzyl, methyl, or ethyl esters of amino acid compounds, may be used to prepare the modified amino acids. The amino add ester, dissolved in a suitable organic solvent such as dimethylformamide, pyridine, or tetrahydrofuran can be reacted with the appropriate amino acid modifying agent at a temperature ranging between 5° C. and 70° C., preferably 25° C., for a period ranging between 7 and 24 hours. The amount of amino acid modifying agent used relative to the amino acid ester is the same as described above for amino acids. This reaction may be carried out with or without a base such as, for example, triethylamine or diisopropylethylamine.

Thereafter, the reaction solvent is removed under negative pressure and the ester functionality is removed by hydrolyzing the modified amino acid ester with a suitable alkaline solution, e.g. 1N sodium hydroxide, at a temperature ranging between 50° C. and 80° C., preferably 70° C., for a period of time sufficient to hydrolyze off the ester group and form the modified amino acid having a free carboxyl group. The hydrolysis mixture is then cooled to room temperature and acidified, e.g. aqueous 25% hydrochloric acid solution, to a pH ranging between 2 and 2.5. The modified amino acid precipitates out of solution and is recovered by conventional means such as filtration or decantation. Benzyl esters may be removed by hydrogenation in an organic solvent using a transition metal catalyst.

The modified amino acid may be purified by recrystallization or by fractionation on solid column supports. Suitable recrystallization solvent systems include acetonitrile, methanol and tetrahydrofuran. Fractionation may be performed on a suitable solid column supports such as alumina, using methanol/n-propanol mixtures as the mobile phase; reverse phase column supports using trifluoroacetic acid/acetonitrile mixtures as the mobile phase; and ion exchange chromatography using water as the mobile phase. When anion exchange chromatography is performed, preferably a subsequent 0-500 mM sodium chloride gradient is employed.

In particular embodiments, modified amino acids having the formula

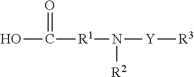

wherein Y is

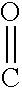

### or SO₂;

R¹ is C₃-C₂₄ alkylene, C₂-C₂₀ alkenylene, C₂-C₂₀ alkynylene, cycloalkylene, or an aromatic, such as arylene;
R² is hydrogen, C₁-C₄ alkyl, or C₂-C₄ alkenyl; and
R³ is C₁-C₇ alkyl, C₃-C₁₀ cycloalkyl, aryl, thienyl, pyrrolo, or pyridyl, and
R³ is optionally substituted by one or more C₁-C₅ alkyl group, C₂-C₄ alkenyl group, F, Cl, OH, OR¹, SO₂, COOH, COOR¹ or, SO₃H;
may be prepared by
reacting in water and the presence of a base a lactam having the formula

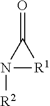

with a compound having the formula R³—Y—X, wherein Y, R¹, R², and R³ are as above and X is a leaving group, A lactam as shown in the above formula can be prepared, for example by the method described in Olah et al., Synthesis, 537-538 (1979).

In particular embodiments, modified amino acids also include an amino acid acylated at its alpha amino group with a fatty acid, which can be represented by the general formula A-X, wherein A is the alpha-amino acid residue and X is a fatty acid attached by acylation to A's alpha-amino group. The amino acids include cationic and non-cationic amino acids. In particular embodiments the term “non-cationic amino acid” refers to an amino acid selected from the group consisting of non-polar hydrophobic amino acids, polar non-charged amino acids, and polar acidic amino acids. In particular embodiments the term “non-cationic amino acid” as used herein refers to amino acids selected from the group consisting of Alanine (Ala), Valine (Val), Leucine (Leu), Isoleucine (Ile), Phenylalanine (Phe), Tryptophan (Trp), Methionine (Met), Proline (Pro), Sarcosine, Glycine (Gly), Serine (Ser), Threonine (Thr), Cysteine (Cys), Tyrosine (Tyr), Asparagine (Asn), and Glutamine (Gin), Aspartic acid (Asp), and Glutamic acid

In particular embodiments, the acylated FA-aa includes an alpha amino acid residue of a non-polar hydrophobic amino acid. In particular embodiments, the acylated FA-aa may be represented by the general formula A-X, wherein A is the amino acid residue of a non-polar hydrophobic amino acid and X is a fatty acid attached by acylation to A's alpha-amino group. In particular embodiments the term “non-polar hydrophobic amino acid” as used herein refers to categorization of amino acids used by the person skilled in the art. In particular embodiments the term “non-polar hydrophobic amino acid” refers to an amino acid selected from the group consisting of Alanine (Ala), Valine (Val), Leucine (Leu), Isoleucine (Ile), Phenylalanine (Phe), Tryptophan (Trp), Methionine (Met), Proline (Pro) and Sarcosine.

In particular embodiments, the acylated FA-aa includes the amino acid residue of a polar non-charged amino acid. In particular embodiments the acylated FA-aa may be represented by the general formula A-X, wherein A is the amino acid residue of a polar non-charged amino acid and X is a fatty acid attached by acylation to A's alpha-amino group. In particular embodiments the term “polar non-charged amino acid” as used herein refers to categorization of amino acids used by the person skilled in the art. In particular embodiments the term “polar non-charged amino acid” refers to an amino acid selected from the group consisting of Glycine (Gly), Serine (Ser), Threonine (Thr), Cysteine (Cys), Tyrosine (Tyr), Asparagine (Asn), and Glutamine (Gin).

In particular embodiments, the acylated FA-aa includes the amino acid residue of a polar acidic amino acid. In particular embodiments, the acylated FA-aa may be represented by the general formula A-X, wherein A is the amino acid residue of a polar acidic amino acid and X is a fatty acid attached by acylation to A's alpha-amino group. In particular embodiments, the term “polar acidic amino acid” as used herein refers to categorization of amino acids used by the person skilled in the art. In particular embodiments, the term “polar acidic amino acid” refers to an amino acid selected from the group consisting of Aspartic acid (Asp) and Glutamic acid (Glu).

In particular embodiments, the amino acid residue of the acylated FA-aa includes the amino acid residue of an amino acid that is not encoded by the genetic code. Modifications of amino acids by acylation may be readily performed using acylation agents known in the art that react with the free alpha-amino group of the amino acid.

In particular embodiments, the alpha-amino acids or the alpha-amino acid residues herein are in the L-form unless otherwise stated.

In particular embodiments, the amino acid residue is in the free acid form and/or a salt thereof, such as a sodium (Na+) salt thereof.

Exemplary embodiments of acylated FA-aas may be represented by the general Fa-aa formula I:

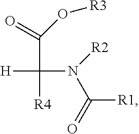

wherein R1 is an alkyl or aryl group including 5 to 19 carbon atoms; R2 is H (i.e. hydrogen), CH₃ (i.e. methyl group), or covalently attached to R4 via a (CH₂)₃ group; R3 is H or absent; and R4 is an amino acid side chain or covalently attached to R2 via a (CH₂)₃ group; or a salt thereof.

The FA-aa can be acylated with a fatty acid including a substituted or unsubstituted alkyl group consisting of 5 to 19 carbon atoms. In particular embodiments, the alkyl group consists of 5 to 17 carbon atoms. In particular embodiments, the alkyl group consists of 5-15 carbon atoms. In particular embodiments the alkyl group consists of 5-13 carbon atoms. In particular embodiments the alkyl group consists of 6 carbon atoms.

In particular embodiments, the acylated FA-aa is soluble at intestinal pH values, particularly in the range pH 5.5 to 8.0, such as in the range pH 6.5 to 7.0. In particular embodiments, the acylated FA-aa is soluble below pH 9.0.

In particular embodiments, the acylated FA-aa has a solubility of at least 5 mg/mL. In particular embodiments, the acylated FA-aa has a solubility of at least 10 mg/mL. In particular embodiments, the acylated FA-aa has a solubility of at least 20 mg/mL. In particular embodiments, the acylated FA-aa has a solubility of at least 30 mg/mL. In particular embodiments, the acylated FA-aa has a solubility of at least 40 mg/mL. In particular embodiments, the acylated FA-aa has a solubility of at least 50 mg/mL. In particular embodiments, the acylated FA-aa has a solubility of at least 60 mg/mL. In particular embodiments, the acylated FA-aa has a solubility of at least 70 mg/mL. In particular embodiments, the acylated FA-aa has a solubility of at least 80 mg/mL. In particular embodiments, the acylated FA-aa has a solubility of at least 90 mg/mL. In particular embodiments, the acylated FA-aa has a solubility of at least 100 mg/mL. In particular embodiments, solubility of the acylated FA-aa is determined in an aqueous solution at a pH value 1 unit above or below pKa of the FA-aa at 37° C. In particular embodiments, solubility of the acylated FA-aa is determined in an aqueous solution at pH 8 at 37° C. In particular embodiments, solubility of the acylated FA-aa is determined in an aqueous solution at a pH value 1 unit above or below pl of the FA-aa at 37° C. In particular embodiments, solubility of the acylated FA-aa is determined in an aqueous solution at a pH value 1 units above or below pl of the FA-aa at 37° C., wherein said FA-aa two or more ionisable groups with opposite charges. In particular embodiments, solubility of the FA-aa is determined in an aqueous 50 mM sodium phosphate buffer, pH 8.0 at 37° C.

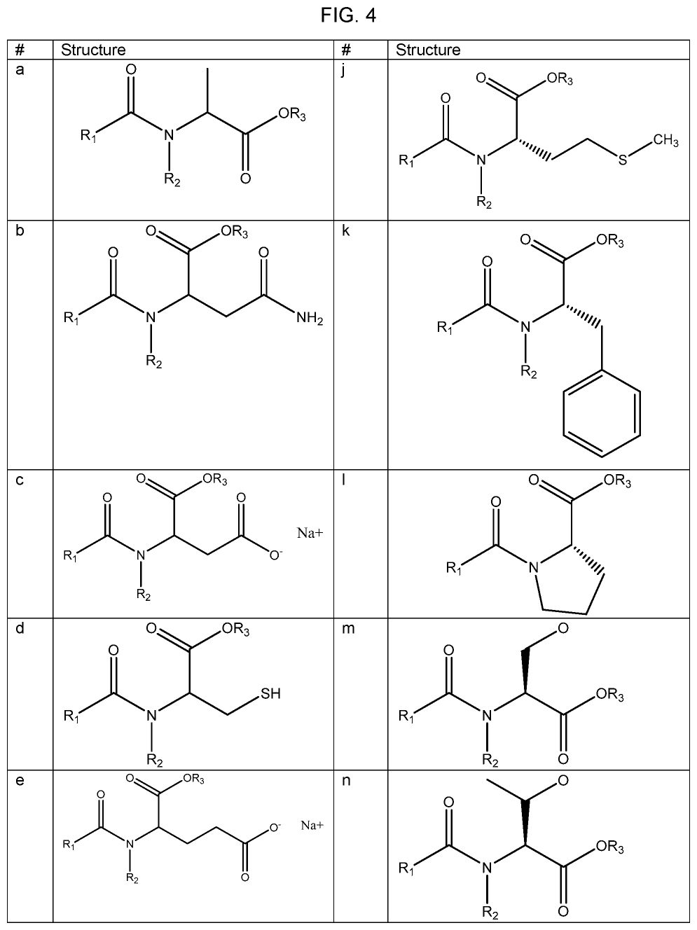

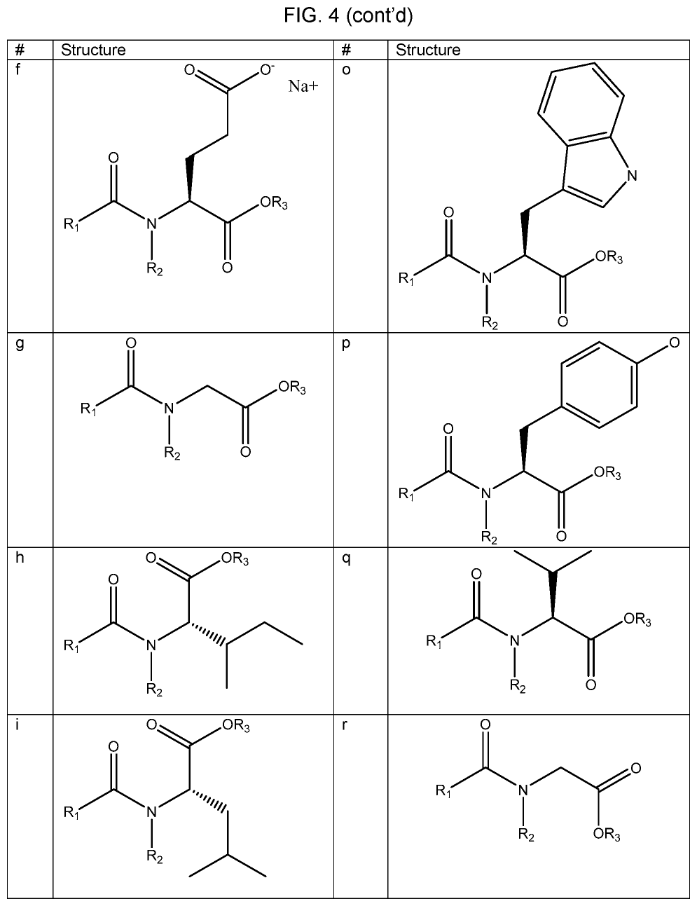

In particular embodiments the acylated FA-aa is selected from the group consisting of formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH₃ (i.e. methyl group), and R3 is H; or a salt or the free acid form thereof. Formulas (a), (b), (c), (d), (e), (f), (g), (h), (i), (j), (k), (l), (m), (n), (o), (p), (q), and (r) are provided in FIG. 4.

In particular embodiments, the acylated FA-aa can be selected from one or more of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophane, sodium N-dodecanoyl valinate, N-dodecanoyl-L-valine, sodium N-dodecanoyl sarcosinate, N-dodecanoyl-L-sarcosine, sodium N-oleoyl sarcosinate, sodium N-decyl leucine, sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decanoyl isoleucinate, N-decanoyl-L-isoleucine, sodium N-decanoyl methioninate, N-decanoyl-L-methionine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophane, sodium N-decanoyl sarcosinate, N-decanoyl-L-Sarcosine, N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl tyrosinate, N-decanoyl-L-tyrosine, sodium N-dodecanoyl asparaginate, sodium N-dodecanoyl glutamic acid, N-dodecanoyl-L-glutamic acid, sodium N-decanoyl glutamic acid, N-decanoyl-L-glutamic acid, Amisoft HS-11 P (sodium Stearoyl Glutamate, Amisoft MS-11 (sodium Myristoyl Glutamate), Amisoft LS-11 (sodium Dodecanoyl Glutamate), Amisoft CS-11 (sodium Cocoyl Glutamate), sodium N-cocoyl glutamate, Amisoft HS-11 P, Amisoft HS-11 P (sodium N-stearoyl glutamate), (sodium N-myristoyl glutamate)), (sodium N-dodecanoyl glutamate), and Amisoft HS-11 P.

The following acylated FA-aas are commercially available:

| | | Provider |
|---|---|---|
| Brand Name | Chemical Name | (per Apr. 14, 2011) |
| Hamposyl L-95 | sodium N-dodecanoyl sarcosinate | Chattem Chemicals |
| Hamposyl O | sodium N-oleoyl sarcosinate | Chattem Chemicals |
| Hamposyl C | sodium N-cocoyl sarcosinate | Chattem Chemicals |
| Hamposyl L-30 | sodium N-dodecanoyl sarcosinate | Chattem Chemicals |
| Amisoft HS-11 P | sodium N-stearoyl glutamate | Ajinomoto |
| Amisoft LS-11 | sodium N-dodecanoyl glutamate | Ajinomoto |
| Amisoft CS-11 | sodium N-cocoyl glutamate | Ajinomoto |
| Amisoft MS-11 | sodium N-myristoyl glutamate | Ajinomoto |
| Amilite GCS-11 | sodium N-cocoyl glycinate | Ajinomoto |

In particular embodiments the terms “fatty acid N-acylated amino acid”, “fatty acid acylated amino acid”, or “acylated amino acid” are used interchangeably herein and refer to an amino acid that is acylated with a fatty acid at its alpha-amino group.

Particular embodiments utilize cannabinoids with low solubility, or very low solubility. Particular embodiments utilize cannabinoids that are essentially water insoluble. In particular embodiments, low solubility can refer to less than 0.2 mg/ml solubility in water or an aqueous solution, or less than 0.1 mg/ml solubility in water or an aqueous solution. In particular embodiments, solubility in water is defined as low to insoluble by the United States pharmacopeia (USP 32) according to the amount of water necessary for the dissolution of one part of solute: Low solubility: 100 to 1000 parts of water necessary for dissolution of one part of solute; very low solubility: 1000 to 10 000 parts of water necessary; essentially water insoluble more than 10 000 parts of water necessary. At a basic pH, however, SNAC and other modified amino acids and FA-aas described herein are water soluble. Thus, the administration benefits, as described herein could not be reasonably predicted.

In particular embodiments, N-acylated fatty amino acids act as absorption enhancing agents, thereby creating an administration benefit. Absorption enhancing agents refer to compounds that promote gastrointestinal absorption. Absorption enhancing agents can improve drug absorption by improving the solubility of the drug in the gastrointestinal tract or by enhancing membrane penetration, as compared to a formulation that does not include the absorption enhancing agents. Additional examples of absorption enhancing agents include surfactants, detergents, azones, pyrrolidones, glycols or bile salts.

In particular embodiments, N-acylated fatty amino acids act as bioavailability enhancing agents. Bioavailability refers to the fraction of active ingredient that is actually absorbed by a subject and reaches the bloodstream. In particular embodiments, bioavailability enhancing agents increase the fraction of active ingredient in the bloodstream or result in detection of active ingredient in the bloodstream earlier in time, as compared to a formulation that does not include the bioavailability enhancing agent.

In particular embodiments, additional administration benefits created by absorption enhancing agents and/or bioavailability enhancing agents include faster onset of action, higher peak concentrations, faster time to peak concentrations, increased subjective therapeutic efficacy, and/or increased objective therapeutic efficacy as compared to a control synthetic cannabinoid composition or oral formulation based on the same, similar in all aspects but for inclusion of the absorption enhancing agents and/or bioavailability enhancing agents.

Embodiments utilizing absorption enhancing agents and/or bioavailability enhancing agents (e.g., and in particular embodiments, N-acylated fatty amino acids) can be beneficial because many oral cannabinoid compositions designed to address various physiological conditions are inadequate because they are characterized by a delayed onset of action, and low bioavailability. Delayed onset of action presents challenges in clinical indications that require rapid therapeutic effect (e.g. pain and migraine); and low bioavailability requires patients to ingest significantly higher doses than would be required by alternative dosing forms (e.g. smoking, vaping). Particular embodiments disclosed herein provide oral formulations including the compositions provided herein with improved bioavailability and shorter time to onset of therapeutic effect.

As stated, in particular embodiments, N-acylated fatty amino acids act as subjective therapy enhancing agents. Subjective therapy enhancement refers to a noticeable alleviation of a symptom, as perceived by a subject. In particular embodiments, subjective therapy enhancing agents increase the alleviation of a symptom or alleviate a symptom more quickly, as compared to a formulation that does not include the subjective therapy enhancing agent.

In particular embodiments, N-acylated fatty amino acids act as objective therapy enhancing agents. Objective therapy enhancement refers to alleviation of a clinical measure, such as a nutritional deficiency detected by a blood or saliva assay or a test of wellness, as administered by a physician. In particular embodiments, objective therapy enhancing agents increase the alleviation of an objective clinical measure or result in alleviation more quickly, as compared to a formulation that does not include the objective therapy enhancing agent.

Particular embodiments include synthetic cannabinoids and an absorption enhancing agent and/or bioavailability enhancing agent. These embodiments can allow more rapid cannabinoid absorption and higher bioavailability compared to cannabinoids ingested by currently available oral dosage forms.

In particular embodiments, carriers disclosed herein create administration benefits selected from: increased absorption, increased bioavailability, faster onset of action, higher peak concentrations, faster time to peak concentrations, increased subjective therapeutic efficacy, increased objective therapeutic efficacy, improved taste, and improved mouthfeel. Administration benefits related to increased absorption, increased bioavailability, faster onset of action, higher peak concentrations, and faster time to peak concentrations, can alleviate adverse conditions more rapidly (for example, alleviation of pain). “Mouthfeel” refers to non-taste-related aspects of the pleasantness experienced by a person while ingesting (e.g., chewing or swallowing) an oral dosage form. Aspects of mouthfeel include the hardness and brittleness of a composition, whether the composition is chewy, gritty, oily, creamy, watery, sticky, easily dissolved, astringent, effervescent, and the like, and the size, shape, and form of the composition (tablet, powder, gel, etc.). In particular embodiments, the administration benefit is a dose-dependent administration benefit. A dose-dependent administration benefit may refer to an administration benefit that occurs when the carrier is within a range of doses, or a range of relative doses (relative to an active ingredient). In particular embodiments, the dose-dependent administration benefits occur when the carrier is at a dose that is one to one hundred times or one to twenty times the dose of an active ingredient.

In particular embodiments, the compositions provided herein can be manufactured for administration to a subject by adding one or more synthetic cannabinoid, a carrier that provides an administration benefit, and one or more excipients, mixing, suspending, dissolving, blending, granulating, tableting, encapsulating, or performing other dosage-form-specific procedures, followed by packaging. For clarity, carriers contribute to providing an administration benefit. Excipients can, but need not, contribute to an administration benefit.

Particular embodiments include compositions prepared as oral formulations. Exemplary oral formulations include capsules, coated tablets, edibles, elixirs, emulsions, gels, gelcaps, granules, gums, juices, liquids, oils, pastes, pellets, pills, powders, rapidly-dissolving tablets, sachets, semi-solids, sprays, solutions, suspensions, syrups, tablets, etc.

Exemplary excipient classes include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the oral formulations to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of oral formulations. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycine.

Disintegrants also may be included in the oral formulations in order to facilitate dissolution. Disentegrants, including permeabilising and wicking agents, are capable of drawing water or saliva up into the oral formulations which promotes dissolution from the inside as well as the outside of the oral formulations. Such disintegrants, permeabilising and/or wicking agents that may be used include starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose. Dissolution of the oral formulations may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Exemplary emulsifiers include acacia and lecithin.

Flavorants are natural or artificial compounds used to impart a pleasant flavor and often odor to oral formulations. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize oral formulation weight variation. Exemplary glidants include silicon dioxide, colloidal or fumed silica, magnesium stearate, calcium stearate, stearic acid, cornstarch, and talc.

Lubricants are substances used in oral formulations that reduce friction during composition compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Exemplary preservatives include methyl p-hydroxybenzoates, propyl p-hydroxybenzoates, and sorbic acid.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

Particular embodiments include swallowable compositions. Swallowable compositions are those that do not readily dissolve when placed in the mouth and may be swallowed whole without chewing or discomfort. U.S. Pat. Nos. 5,215,754 and 4,374,082 describe methods for preparing swallowable compositions. In particular embodiments, swallowable compositions may have a shape containing no sharp edges and a smooth, uniform and substantially bubble free outer coating.

To prepare swallowable compositions, each of the ingredients may be combined in intimate admixture with a suitable carrier according to conventional compounding techniques. In particular embodiments of the swallowable compositions, the surface of the compositions may be coated with a polymeric film. Such a film coating has several beneficial effects. First, it reduces the adhesion of the compositions to the inner surface of the mouth, thereby increasing the subject's ability to swallow the compositions. Second, the film may aid in masking the unpleasant taste of certain ingredients. Third, the film coating may protect the compositions from atmospheric degradation. Polymeric films that may be used in preparing the swallowable compositions include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In particular embodiments, the oral formulations may include chewable compositions. Chewable compositions are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable compositions with improved mouthfeel. U.S. Pat. No. 5,965,162, describes kits and methods for preparing comestible units which disintegrate quickly in the mouth, especially when chewed.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

Sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Fondant or gums such as gelatin, agar, arabic gum, guar gum, and carrageenan may be added to improve the chewiness of the compositions. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable compositions. By “enhance the texture” it is meant that the alkyl polysiloxane improves one or more of the stiffness, the brittleness, and the chewiness of the chewable composition, relative to the same preparation lacking the alkyl polysiloxane. By “enhance the mouthfeel” it is meant that the alkyl polysiloxane reduces the gritty texture of the chewable composition once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the back bone. Depending upon their grade, they can further include silica gel. Alkyl polysiloxanes are generally viscous oils. Exemplary alkyl polysiloxanes that can be used in swallowable, chewable or dissolvable compositions include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than 90.5% by weight (CH₃)₃—Si{OSi(CH₃)₂}CH₃ in admixture with 4.0% to 7.0% by weight SiO₂.

To prevent the stickiness that can appear in some chewable compositions and to facilitate conversion of the active ingredients to emulsion or suspension upon taking, the compositions, may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the administered formulations. If the level of emulsifier is lower or higher, in particular embodiments, an emulsification cannot be realized, or wax value will rise.

Liquid preparations for oral administration may take the form of, for example, solutions, syrups or suspensions, or they may be presented as a dry product for reconstitution with water or other suitable vehicles before use.

In addition to those described above, any appropriate fillers and excipients may be utilized in preparing the swallowable, chewable and/or dissolvable compositions or any other oral formulation described herein so long as they are consistent with the described objectives.

Oral formulations also include edibles. Edibles refer to any product that can be consumed as a food or a drink. In some cases, edibles can be made by infusion of the composition provided herein into a foodstuff. Examples of edible foods appropriate for use include candy, a candy bar, bread, a brownie, cake, cheese, chocolate, cocoa, a cookie, gummy candy, a lollipop, a mint, a pastry, peanut butter, popcorn, a protein bar, rice cakes, yogurt, etc. While technically not edible, gums can also be used. Examples of edible drinks include beer, juice, flavored milk, flavored water, liquor, milk, punch, a shake, soda, tea, and water. In particular embodiments, edibles are made by combining a synthetic cannabinoid/carrier composition with ingredients used to make an edible. Examples include butters and oils. Exemplary oils include coconut oil, grape seed oil, olive oil, palm oil, papaya seed oil, peanut oil, sesame oil, sprouted wheat oil, wheat germ oil, or any combination thereof.

Oral formulations can be individually wrapped or packaged as multiple units in one or more packages, cans, vials, blister packs, or bottles of any size. Doses are sized to provide therapeutically effective amounts.

In particular embodiments, the oral formulations include a synthetic cannabinoid (e.g., nabilone, CBD and/or THC) of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include carrier of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include excipient of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, 10 g of the composition provided herein may be used in 150 ml of water. This may give an effective concentration of between 1 and 99% (w/w), between 2 and 80% (w/w), or between 5 and 50% (w/w) of the composition.

Excipients are commercially available from companies such as Aldrich Chemical Co., FMC Corp, Bayer, BASF, Alexi Fres, Witco, Mallinckrodt, Rhodia, ISP, and others.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990. Moreover, formulations can be prepared to meet sterility, pyrogenicity, general safety, and purity standards as required by U.S. FDA and/or other relevant foreign regulatory agencies.

Exemplary formulation methods. Solution formulation. Synthetic cannabinoids and one or more N-acylated fatty amino acids are combined in an aqueous/organic solvent mixture. The resulting blend is stirred vigorously for an hour. If solution is incomplete, a surfactant can be added and stirring can be continued to prepare the final formulation.

Suspension formulation. Synthetic cannabinoids and one or more N-acylated fatty amino acids are combined in water, an aqueous/organic solvent mixture or an organic solvent mixture. The resulting blend can be stirred to effect suspension.

Solution formulation. Synthetic cannabinoids and any one or more absorption enhancing agents are combined in an aqueous/organic solvent mixture. The resulting blend is stirred vigorously for an hour. If solution is incomplete, a surfactant can be added and stirring can be continued to prepare the final formulation.

Suspension formulation. Synthetic cannabinoids and any one or more absorption enhancing agents are combined in water, an aqueous/organic solvent mixture or an organic solvent mixture. The resulting blend can be stirred to effect suspension.

Gelcap composition. A suspension formulation or solution formulation can be filled into a gelcap to contain up to 1 g of synthetic cannabinoid. The gelcap can be treated with an enteric coat or used without a coating.

Tablet/capsule composition. The solution formulation and the suspension formulation can be dried by evaporation, lyophilization, or spray drying. The resultant dry product can be combined with tableting excipients and compressed into tablets or caplets to contain up to 1 g of synthetic cannabinoid. Alternatively, the dry product can be filled into capsules.

The compositions disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing therapeutically effective amounts. Therapeutically effective amounts include those that provide effective amounts, prophylactic treatments, and/or therapeutic treatments.

An “effective amount” is the amount of the composition necessary to result in a desired physiological change in a subject. Effective amounts are often administered for research purposes. Representative effective amounts disclosed herein can reduce pain perception in an animal model (neuropathic pain, acute pain, visceral pain), stimulate appetite in an animal model, reduce seizures (e.g., epileptic seizures) in an animal model, reverse bone loss in an animal model, relieve migraine (vasoconstrict cranial blood vessels) in an animal model, treat addiction in an animal model, reduce anxiety in an animal model, and/or reduce symptoms of asthma in an animal model.

A “prophylactic treatment” includes a treatment administered to a subject who does not display signs or symptoms of a disease or nutritional deficiency, or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As one example of a prophylactic treatment, an oral formulation disclosed herein can be administered to a subject who is at risk of developing a migraine headache. An effective prophylactic treatment of a migraine headache occurs when the number of migraines per month experienced by a subject is reduced by at least 10% or in particular embodiments, by 25%.

As another example of a prophylactic treatment, an oral formulation disclosed herein can be administered to a subject who is at risk of having an epileptic seizure. An effective prophylactic treatment of epileptic seizures occurs when the number of seizures per month is reduced by at least 10% or in particular embodiments, by 25%.

As another example of a prophylactic treatment, an oral formulation disclosed herein can be administered to a subject who is at risk of suffering from neuropathic pain. An effective prophylactic treatment of neuropathic pain occurs when the occurrence of the neuropathic pain is reduced by at least 10%, or in particular embodiments, by 25% as measured by a standard subjective or objective pain assessment.

As another example of a prophylactic treatment, an oral formulation disclosed herein can be administered to a subject who is at risk of developing breakthrough pain. An effective prophylactic treatment of breakthrough pain occurs when the occurrence of breakthrough pain is reduced by 10%, and in particular embodiments, by 25% by a standard subjective or objective pain assessment.

As another example of a prophylactic treatment, an oral formulation disclosed herein can be administered to a subject who is at risk of developing chemotherapy induced nausea and vomiting (CINV). An effective prophylactic treatment of CINV occurs when CINV is reduced by 10%, and in particular embodiments, by 25% measured by a standard subjective or objective CINV assessment.

As an example of a prophylactic treatment of a nutritional deficiency, an oral formulation disclosed herein can be administered to a subject who is at risk of developing rickets from insufficient vitamin C, anemia from insufficient dietary iron, and/or bone loss from insufficient calcium. An effective prophylactic treatment of these conditions occurs when the conditions are avoided or delayed due to nutritional supplementation with an oral formulation disclosed herein.

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, an oral formulation disclosed herein can be administered to a subject who has a migraine headache. An effective therapeutic treatment of the migraine headache occurs when the severity of the headache is reduced or relieved completely and/or the headache resolves more quickly measured by a standard subjective or objective headache assessment.

Another example of a therapeutic treatment includes administration of an oral formulation disclosed herein to a subject experiencing CINV. A therapeutic treatment of CINV occurs when the vomiting is reduced or ceases (or ceases more quickly) and the nausea is relieved measured by a standard subjective or objective CINV assessment.

Another example of a therapeutic treatment, includes administration of an oral formulation disclosed to a subject who has osteoporosis. An effective therapeutic treatment of osteoporosis occurs when bone density has increased by 10% and in particular embodiments, by 25%.

Another example of a therapeutic treatment includes administration of an oral formulation disclosed herein to a subject who has anxiety. An effective therapeutic treatment of anxiety occurs when the severity of the anxiety is reduced or relieved completely and/or more quickly measured by a standard subjective or objective anxiety assessment.

Another example of a therapeutic treatment includes administration of an oral formulation disclosed herein to a subject who has multiple sclerosis. An effective therapeutic treatment of multiple sclerosis occurs when the score in a standard walk test improves by 10% and in particular embodiments, by 25%.

As one example of a therapeutic treatment of a nutritional deficiency, an oral formulation disclosed herein can be administered to a subject who has rickets from insufficient vitamin C, anemia from insufficient dietary iron, and/or bone loss from insufficient calcium. An effective therapeutic treatment of these conditions occurs when the conditions are reduced or resolved due to nutritional supplementation with an oral formulation disclosed herein.

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

Useful doses can range from 0.1 to 5 μg/kg or from 0.5 to 1 μg/kg. In other non-limiting examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In other non-limiting examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more.

In particular embodiments, useful doses include weight of a synthetic cannabinoid or active ingredient per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, or more of synthetic cannabinoid or active ingredient per body weight of a subject.

In particular embodiments, useful doses include weight of carrier (e.g., SNAC) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more of carrier per body weight of a subject. In particular embodiments, useful doses of carrier (e.g., SNAC) may provide an administration benefit.

In particular embodiments, total dose volume can range from 0.25 mL to 30 mL or from 0.5 mL to 20 mL. In particular embodiments, a total dose volume can include 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, or more.

Dose concentration can be expressed as weight of synthetic cannabinoid or active ingredient per dose volume (e.g., mg active pharmaceutical ingredient (API)/mL). In particular embodiments, dose concentration can range from 1 mg/mL to 100 mg/mL or from 5 mg/mL to 50 mg/mL. In particular embodiments, a dose concentration can include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Dose concentration can be expressed as weight of carrier (e.g., SNAC) per dose volume (e.g., mg SNAC/mL). In particular embodiments, dose concentration can range from 1 mg/mL to 500 mg/mL or from 50 mg/mL to 300 mg/mL. In particular embodiments, a dose concentration can include 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, or more.

In particular embodiments, the ratio of carrier to cannabinoid or active ingredient (w/w) can range from 1:1 to 100:1 or from 1:1 to 20:1. In particular embodiments, the ratio can include 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, or more. In particular embodiments, the ratio can be 10:1. In particular embodiments, a ratio of a carrier to a cannabinoid (w/w) of between 1:1 and 100:1 may provide an administration benefit. In particular embodiments, a ratio of a carrier to a cannabinoid (w/w) of between 1:1 and 20:1 may provide an administration benefit.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more active agent(s) can be administered simultaneously or within a selected time window, such as within 10 minutes, 1 hour, 3 hour, 10 hour, 15 hour, 24 hour, or 48 hour time windows or when the complementary active agent(s) is within a clinically-relevant therapeutic window.

The term “halogen” or “halo” or “halogen atom” refers fluorine, chlorine, bromine, or iodine.

The term “alkyl group” refers to a saturated branched or straight chain hydrocarbon radical or group. “Alkyl” includes methyl, ethyl, propyl, isopropyl, n-butyl, t-butyl, pentyl, hexyl, isohexyl, neohexyl, heptyl, octyl, nonyl, decyl, undecyl, dodecyl, tridecyl, tetradecyl, pentadecyl, hexadecyl, heptadecyl, octadecyl, and the like.

The term “aryl group” refers to an unsaturated, conjugated π electron monocyclic or polycyclic hydrocarbon ring system radical or linking group of 6 or more carbon atoms. An aryl radical is derived by the removal of one hydrogen atom from a single carbon ring atom. An arylene linking group is derived by the removal of two hydrogen atoms each of two carbon ring atoms. “Aryl” includes phenyl, napthyl, phenanthryl, anthryl, naphthalenyl, azulenyl, anthracenyl and the like.

The term “heteroaryl group” refers to an aryl having one or more nitrogen atom(s), oxygen atom(s), and/or sulfur atom(s).

The term “cycloalkyl group” refers to a saturated or partially unsaturated monocyclic, polycyclic or bridged hydrocarbon ring system radical or linking group. “Cycloalkyl” includes cyclopropyl, cyclobutyl, cyclopentyl, cyclohexyl, cyclohexenyl, cycloheptyl, cyclooctyl or cyclooxtyl,

The term “heterocyclic group” refers to a saturated or unsaturated, cyclic or polycyclic hydrocarbon based chain including one or more heteroatoms chosen from O, S, and N. The term “heterocyclic group” also includes a “heteroaryl group.”

The term “heteroaryl group” refers to an aromatic heterocyclic group, such as a cyclic or polycyclic aromatic hydrocarbon based chain, including one or more heteroatoms chosen from O, S and N. Accordingly, a heteroaryl group is an example of a heterocyclic group. As an example, the aromatic hydrocarbon based chain can include 3 to 10 carbons and/or heteroatoms and one or more double bonds. The polycyclic aromatic hydrocarbon based chain includes two or more fused aromatic rings.

The term “alkenyl group” means carbon chains which contain at least one carbon-carbon double bond, and which may be linear or branched or combinations thereof. Examples of “alkenyl” include vinyl, allyl, isopropenyl, pentenyl, hexenyl, heptenyl, 1-propenyl, 2-butenyl, 2-methyl-2-butenyl, and the like.

The term “aralkyl group” refers a group in which an “alkyl group” is replaced with an “aryl group”. For example, benzyl, phenylethyl (e.g., 1-phenylethyl, 2-phenylethy), phenylpropyl (e.g., 1-phenylpropyl, 2-phenylpropyl, 3-phenylpropyl), naphthylmethyl (e.g., 1-naphthylmethyl, 2-naphthylmethyl), and the like.

The term “alkynyl group” refers carbon chains which contain at least one carbon-carbon triple bond, and which may be linear or branched or combinations thereof. Examples of “alkynyl” include ethynyl, propargyl, 3-methyl-1-pentynyl, 2-heptynyl, propynyl, butynyl, 2-pentynyl, 3-pentynyl, 2-hexynyl, 3-hexynyl, hepynyl, and the like.

The term “alkylene group” refers to a saturated branched or straight chain hydrocarbon linking group of carbon atoms, whereby the linking group is derived by the removal of one hydrogen atom each from two carbon atoms. “Alkylene” includes methylene, ethylene, propylene, tetramethylene, trimethylene, isopropylene, n-butylene, t-butylene, pentylene, hexylene, heptylene and the like.

The term “alkenylene group” refers to a straight or branched alkylene having one or more double bonds. For example, vinylene, 1-propenylene, allylene, propenylene, isopropenylene, 1-butenylene, 2-butenylene, 3-butenylene, 2-pentenylene, 1,3-butadienylene, 3-methyl-2-butenylene, and the like can be listed. When the number of carbons is specified, the “alkenylene”, which has the number of carbons within the ranges of the number, is meant.

The different groups described above can be optionally substituted, for example, with a halogen, a hydroxyl group, an amino group, an amide group, a cyano group, a nitro, an alkyl group, an alkoxy group, an alkenyl group, a carboxyl group, an aryl group, a heterocyclic group, or a sulfonyl group.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

Exemplary Embodiments.
1. A composition including one or more synthetic cannabinoids or one or more salts thereof and one or more N-acylated fatty amino acids or salts thereof.
2. The composition of embodiment 1, wherein the one or more synthetic cannabinoids include Δ9-Tetrahydrocannabinol (THC) cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), or tetrahydrocannabivarinic acid (THCVA).
3. The composition of embodiment 1, wherein the one or more synthetic cannabinoids include a derivative and/or analog of a synthetic cannabinoid of embodiment 2 or a mixture thereof.
4. The composition of any of embodiments 1-3, wherein the one or more synthetic cannabinoids include 3-carbamoyl-2-pyridone or its derivatives and/or analogs; pyrimidine derivatives and/or analogs; carenadiol or its derivatives and/or analogs; cannabinoid carboxylic acids or their derivatives and/or analogs; pyrido[3,2-E][1,2,4]triazolo[4,3-C]pyrimidine or its derivatives and/or analogs; tetrahydro-pyrazolo[3,4-C] pyridine or its derivatives and/or analogs; bicyclo[3.1.1]heptan-2-one cannabinoid or its derivatives and/or analogs; resorcinol or its derivatives and/or analogs; dexanbinol compounds or their derivatives and/or analogs; cannabimimetic lipid amide compounds or their derivatives and/or analogs; nabilone or its derivatives and/or analogs; 2-oxoquinolone compounds or their derivatives and/or analogs disclosed; or 3,4-diaryl-4,5-dihydro-(h)-pyrazole-1-carboxamide or its derivatives and/or analogs.
5. The composition of any of embodiments 1-4, wherein the one or more N-acylated fatty amino acids or salts thereof include one or more of Compounds I-XXXV, or Formulas a-s.
6. The composition of any of embodiments 1-4, wherein the one or more N-acylated fatty amino acids or salts thereof include Sodium-N-salicyloyl-8-aminocaprylate, Monosodium 8-(N-salicyloylamino) octanoate, N-(salicyloyl)-8-aminooctanoic add monosodium salt, monosodium N-{8-(2phenoxybenzoyl)amino}octanoate, or sodium 8-[(2-hydroxybenzoyl) amino]octanoate.
7. The composition of any of embodiments 1-4 wherein the one or more N-acylated fatty amino acids or salts thereof include

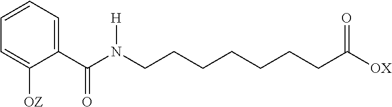

wherein X and Z are independently H, a monovalent cation, a divalent metal cation, or an organic cation.
8. The composition of embodiment 7, wherein the monovalent cation is sodium or potassium.
9. The composition of embodiment 7 or 8, wherein the metal cation is calcium or magnesium.
10. The composition of any of embodiments 7-9, wherein the organic cation is ammonium or tetramethylammonium.
11. The composition of any of embodiments 7-10, wherein X is H.
12. The composition of any of embodiments 7-10, wherein X is a monovalent cation including sodium or potassium.
13. The composition of any of embodiments 7-10, wherein X is a divalent metal cation including calcium or magnesium.
14. The composition of any of embodiments 7-10, wherein X is an organic cation including ammonium or tetramethylammonium.
15. The composition of any of embodiments 7-14, wherein Z is H.
16. The composition of any of embodiments 7-14, wherein Z is a monovalent cation including sodium or potassium.
17. The composition of any of embodiments 7-14, wherein Z is a divalent cation including calcium or magnesium.
18. The composition of embodiment 7, wherein X is H and Z is H.
19. The composition of embodiment 7, wherein X is H and Z is sodium.
20. The composition of embodiment 7, wherein X is sodium and Z is sodium.
21. The composition of any of embodiments 1-20, wherein the one or more N-acylated fatty amino acids or salts thereof provide an administration benefit.
22. The composition of embodiment 21, wherein the administration benefit is a dose-dependent administration benefit.
23. The composition of embodiment 22, wherein the dose-dependent administration benefit is at a dose of 100-200 mg.
24. The composition of embodiment 22, wherein the dose-dependent administration benefit is at a dose concentration of 100 mg/mL to 300 mg/mL N-acylated fatty amino acid or salt thereof.
25. The composition of embodiment 22, wherein the dose-dependent administration benefit is at a dose concentration of 1-500 mg/mL N-acylated fatty amino acid or salt thereof.
26. The composition of embodiment 22, wherein the dose-dependent administration benefit is at a dose concentration of 250 mg/mL N-acylated fatty amino acid or salt thereof.
27. The composition of embodiment 22, wherein the dose-dependent administration benefit of the N-acylated fatty amino acid or salt thereof is at a dose of one to one hundred times the dose of the one or more synthetic cannabinoids.
28. The composition of any of embodiments 1-27, wherein the composition is a pharmaceutical composition.
29. The composition of any of embodiments 1-27, wherein the composition is a nutritional supplement and further includes at least one vitamin and/or mineral.
30. The composition of any of embodiments 1-29, wherein the composition further includes a surfactant, detergent, azone, pyrrolidone, glycol or bile salt.
31. The composition of any of embodiments 1-30, wherein the composition includes a therapeutically effective amount of the one or more synthetic cannabinoids.
32. The composition of embodiment 31 wherein the therapeutically effective amount of the one or more synthetic cannabinoids treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.
33. The composition of any of embodiments 1-32, wherein the composition further includes one or more vitamins or one or more minerals.
34. The composition of any of embodiments 1-32, wherein the composition further includes one or more vitamins and one or more minerals.
35. The composition of embodiment 33 or 34, wherein the one or more vitamins include Vitamin A, Vitamin B1, Vitamin B6, Vitamin B12, Vitamin C, Vitamin D, Vitamin E, or Vitamin K.
36. The composition of embodiment 33 or 34, wherein the one or more minerals include calcium, chromium, iodine, iron, magnesium, selenium, or zinc.
37. An oral formulation, wherein the oral formulation includes the composition of any of embodiments 1-36.
38. An oral formulation, wherein the oral formulation includes the composition of any of embodiments 22-36 and wherein the administration benefit includes one or more of increased absorption of a measured component of the synthetic cannabinoid, increased bioavailability of a measured component of the synthetic cannabinoid, faster onset of action of a measured component of the synthetic cannabinoid, higher peak concentrations of a measured component of the synthetic cannabinoid, faster time to peak concentrations of a measured component of the synthetic cannabinoid, increased subjective therapeutic efficacy, increased objective therapeutic efficacy, improved taste, and improved mouthfeel as compared to a control composition without the N-acylated fatty amino acid or salt thereof.
39. The oral formulation of embodiment 37 or 38, wherein the oral formulation is swallowable or chewable.
40. The oral formulation of embodiment 37 or 38, wherein the oral formulation is liquid or solid.
41. The oral formulation of any of embodiments 37-40, wherein the oral formulation is a solution, suspension, or spray.
42. The oral formulation of any of embodiments 37-40, wherein the oral formulation is a tablet, capsule or sachet.
43. The oral formulation of any of embodiments 37-42, wherein the oral formulation is flavored.
44. A method of treating a subject in need thereof including administering a therapeutically effective amount of a composition of any of embodiments 1-36 or a formulation of any of embodiments 37-43 to the subject thereby treating the subject in need thereof.
45. A method of embodiment 44 wherein the therapeutically effective amount provides an effective amount, a prophylactic treatment, and/or a therapeutic treatment.
46. A method of reducing or eliminating one or more symptoms of a disease or disorder in a human subject,
   wherein said method includes delivering a therapeutically effective amount of a composition of any of embodiments 1-36 or oral formulation of any of embodiments 37-45 to the subject, thereby reducing or eliminating one or more symptoms of the disease or disorder, and
   wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.
47. The method of embodiment 46, wherein the therapeutically effective amount of the composition includes (i) an amount of the N-acylated fatty amino acid or salt thereof and (ii) an amount of the one or more synthetic cannabinoids that are at a ratio (w/w) of between 1:1 and 100:1.
48. A method of preparing a synthetic cannabinoid oral formulation having a faster onset of action, wherein the method includes adding an absorption enhancer to a synthetic cannabinoid and wherein the synthetic cannabinoid oral formulation has a faster onset of action than a synthetic cannabinoid oral formulation without an absorption enhancer.
49. The method of embodiment 48, wherein the absorption enhancer includes an N-acylated fatty amino acid or a salt thereof.
50. The method of embodiment 49, wherein the N-acylated fatty amino acid includes N-[8-(2-hydroxybenzoyl) amino] caprylate.
51. The method of embodiment 48, wherein the absorption enhancer and the synthetic cannabinoid are present at a ratio (w/w) of between 1:1 and 100:1.
52. The method of embodiment 48, wherein the absorption enhancer and the synthetic cannabinoid are present at a ratio (w/w) of between 1:1 and 20:1.

### Example 1

Onset and duration of action of orally administered cannabis/SNAC composition. This study was designed to assess the utility of SNAC in enabling a rapid-acting oral form of cannabis.

Selection of Participants. Six study participants were recruited to ingest cannabis compositions and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 200 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10.

Table 1 shows descriptions of euphoria and dysphoria levels for each scale value.

TABLE 1
Scale Values for Reporting Euphoria and Dysphoria

| Scale Value | Description |
|---|---|
| 0 | No observed effect |
| 1-2 | Mild observed effect; possibly psychological |
| 3-4 | Definite but mild effect |
| 5-6 | Definite substantial effect |
| 7-8 | Strong effect |
| 9-10 | Intense effect |

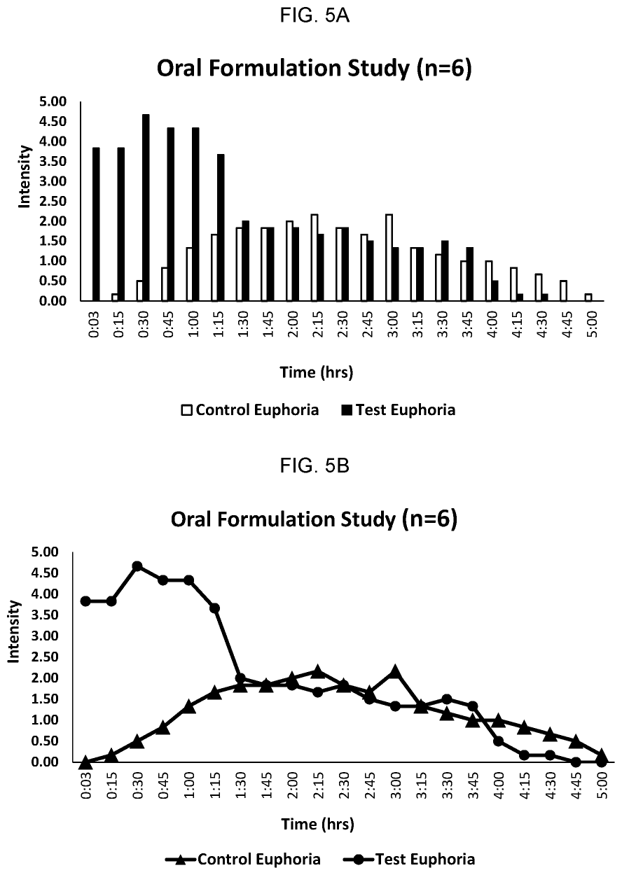

Results. The results shown below are the average scale values obtained for all six participants (also shown in FIGS. 5A and 5B).

TABLE 2
Control Experiment (n = 6)

| Actual | Time | Observed | Observed |
|---|---|---|---|
| Time | from Start | “Euphoria” | “Dysphoria” |
| 12:00 PM | 0:00 | (0-10) | (0-10) |
| 12:15 PM | 0:15 | 0.17 | 0.00 |
| 12:30 PM | 0:30 | 0.50 | 0.00 |
| 12:45 PM | 0:45 | 0.83 | 0.17 |
| 1:00 PM | 1:00 | 1.33 | 0.17 |
| 1:15 PM | 1:15 | 1.67 | 0.50 |
| 1:30 PM | 1:30 | 1.83 | 0.67 |
| 1:45 PM | 1:45 | 1.83 | 0.83 |
| 2:00 PM | 2:00 | 2.00 | 0.50 |
| 2:15 PM | 2:15 | 2.17 | 0.50 |
| 2:30 PM | 2:30 | 1.83 | 0.33 |
| 2:45 PM | 2:45 | 1.67 | 0.33 |
| 3:00 PM | 3:00 | 2.17 | 0.33 |
| 3:15 PM | 3:15 | 1.33 | 0.17 |
| 3:30 PM | 3:30 | 1.17 | 0.00 |
| 3:45 PM | 3:45 | 1.00 | 0.00 |
| 4:00 PM | 4:00 | 1.00 | 0.00 |
| 4:15 PM | 4:15 | 0.83 | 0.00 |
| 4:30 PM | 4:30 | 0.67 | 0.00 |
| 4:45 PM | 4:45 | 0.50 | 0.00 |
| 5:00 PM | 5:00 | 0.17 | 0.00 |

TABLE 3
Test Experiment (n = 6)

| Actual | Time | Observed | Observed |
|---|---|---|---|
| Time | from Start | “Euphoria” | “Dysphoria” |
| 12:00 PM | 0:00 | (0-10) | (0-10) |
| 12:03 PM | 0:03 | 3.83 | 0.67 |
| 12:15 PM | 0:15 | 3.83 | 0.67 |
| 12:30 PM | 0:30 | 4.67 | 0.83 |
| 12:45 PM | 0:45 | 4.33 | 0.50 |
| 1:00 PM | 1:00 | 4.33 | 0.50 |
| 1:15 PM | 1:15 | 3.67 | 0.67 |
| 1:30 PM | 1:30 | 2.00 | 0.17 |
| 1:45 PM | 1:45 | 1.83 | 0.17 |
| 2:00 PM | 2:00 | 1.83 | 0.00 |
| 2:15 PM | 2:15 | 1.67 | 0.00 |
| 2:30 PM | 2:30 | 1.83 | 0.00 |
| 2:45 PM | 2:45 | 1.50 | 0.00 |
| 3:00 PM | 3:00 | 1.33 | 0.17 |
| 3:15 PM | 3:15 | 1.33 | 0.17 |
| 3:30 PM | 3:30 | 1.50 | 1.00 |
| 3:45 PM | 3:45 | 1.33 | 0.00 |
| 4:00 PM | 4:00 | 0.50 | 0.00 |
| 4:15 PM | 4:15 | 0.17 | 0.00 |
| 4:30 PM | 4:30 | 0.17 | 0.00 |
| 4:45 PM | 4:45 | 0.00 | 0.00 |
| 5:00 PM | 5:00 | 0.00 | 0.00 |

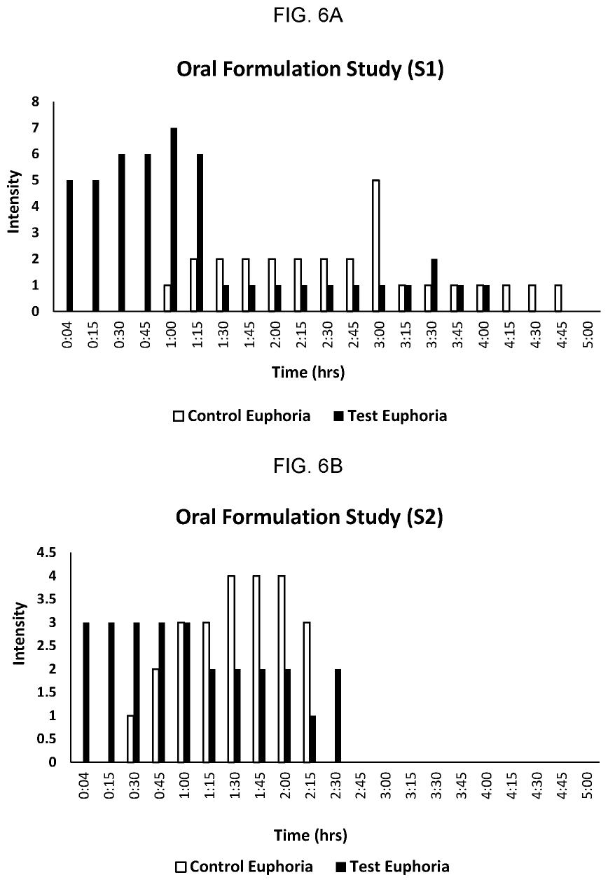

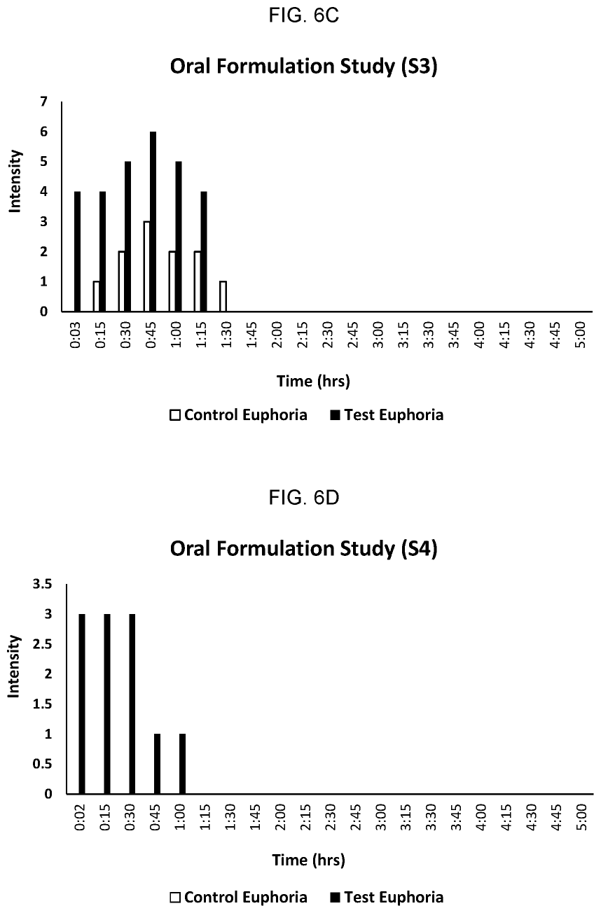

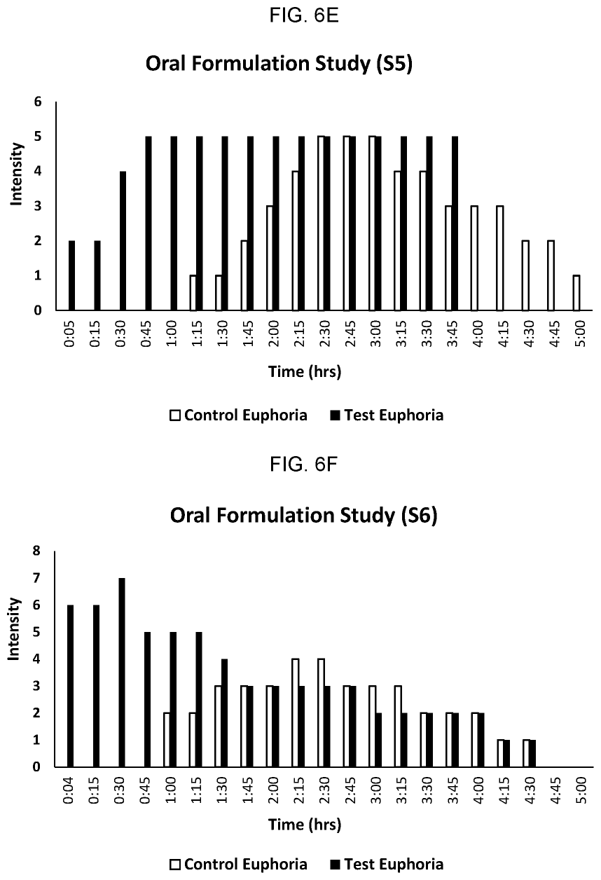

Onset: All six participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes (see FIGS. 6A-6F for individual participant results). By fifteen minutes post-ingestion, the average reported euphoria scale value was 3.8 for the cannabis/SNAC formulation (Test). In contrast, fifteen minutes after ingestion of the cannabis-only formulation (Control), the average reported euphoria scale value was 0.17 (see FIGS. 5A-5B for averages at each time-point).

Intensity: The average peak euphoria scale value after ingestion of the cannabis/SNAC formulation (Test) was 4.7, which occurred thirty minutes post-ingestion. In contrast, the highest average euphoria scale value after ingestion of the cannabis-only formulation (Control) was 2.2, which was at the two hour, fifteen minute time-point (see FIGS. 5A and 5B). Therefore, ingestion of the cannabis/SNAC formulation led to a higher peak intensity of euphoria, which occurred an average of one hour and forty-five minutes faster than when the cannabis-only formulation was ingested. The intensity of observed dysphoria was minimal for both the Test and Control, with a peak average scale value of 0.83 for both experiments.

Duration: The results indicate that the addition of an absorption enhancer does not shorten the duration of action of cannabis.

In summary, adding an absorption enhancer, such as SNAC, in an oral dosage cannabinoid formulation provides faster onset of action and higher intensity of action at peak activity level. Moreover, the absorption enhancer has no effect on the cannabinoid duration of action.

### Example 2

Onset and duration of action of orally administered cannabis/SNAC composition at a low SNAC dose. This study was designed to assess the utility of SNAC in enabling a rapid-acting oral form of cannabis at a low dose.

Selection of Participants. Three study participants were recruited to ingest cannabis compositions and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 100 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

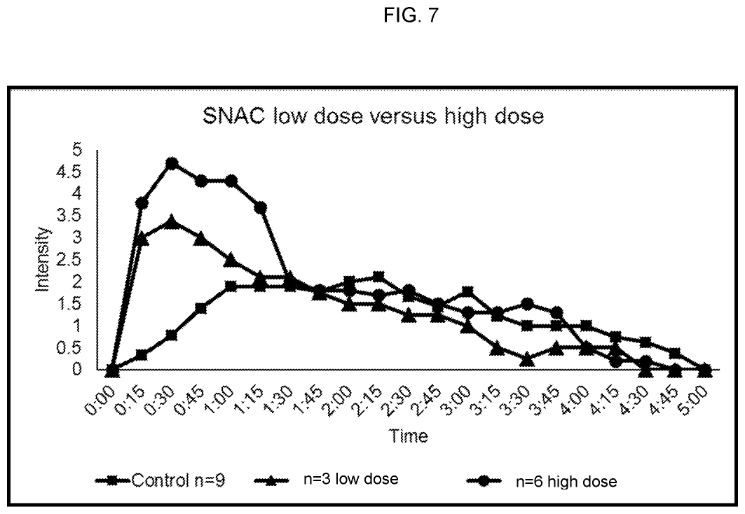

Results. The results are combined with the data from Example 1 and are reported for all participants in FIG. 7.

Onset: All three participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes. By fifteen minutes post-ingestion, the average reported euphoria scale value was 3.0 for the cannabis/SNAC formulation (Test). In contrast, fifteen minutes after ingestion of the cannabis-only formulation (Control), the average reported euphoria scale value was 0.25.

Intensity: The average peak euphoria scale value after ingestion of the cannabis/SNAC formulation (Test) was 3.4, which occurred thirty minutes post-ingestion. In contrast, the highest average euphoria scale value after ingestion of the cannabis-only formulation (Control) was 2.2, which was at the two hour, fifteen minute time-point. Compared to Example 1 where the SNAC dose was 200 mg, the participants in Example 2 ingested only 100 mg of SNAC combined with the same quantity of cannabis used in Example 1. This reduced quantity of SNAC resulted in a reduced cannabis effect demonstrating a clear dose-response relationship between observed cannabis effect (euphoria) and SNAC dose. Consistent with Example 1, ingestion of the cannabis/SNAC formulation led to a higher peak intensity of euphoria, which occurred an average of one hour and forty-five minutes faster than when the cannabis-only formulation was ingested.

Duration: The results indicate that the addition of an absorption enhancer does not shorten the duration of action of cannabis.

In summary, adding an absorption enhancer, such as SNAC, in an oral dosage formulation of cannabinoid provides faster onset of action and higher intensity of action at peak activity level of the cannabinoid. Moreover, the absorption enhancer has no effect on the duration of action. The varying quantity of SNAC produces a clear dose-response relationship between observed cannabinoid effect (euphoria) and SNAC dose.

### Example 3

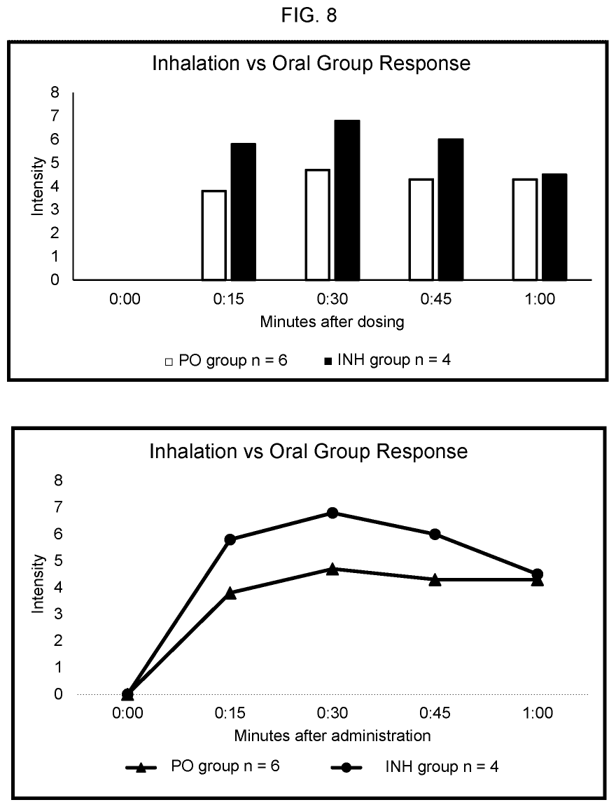

Inhalation versus oral group response (FIG. 8). Comparison of the pharmacodynamic response to inhaled and oral cannabis measured as subject-reported euphoria. Both the oral and inhaled groups reported similar time to peak effect (15-30 minutes). This is very surprising because oral dosage forms of cannabinoids are traditionally characterized by a very slow time to peak effect (up to 4 hours).

### Example 4

Summary of cannabis/SNAC oral rat pharmacokinetic (PK) study. The study was designed to characterize the pharmacokinetic profile of cannabis extract containing 56% THC/CBD in a 1:1 ratio (by weight) with and without the excipient, SNAC, following a single oral gavage administration to rats. In this study two doses of cannabis and SNAC and two ratios of cannabis to SNAC were tested. The experimental design is presented in Table 4 below.

TABLE 4
Experimental design.

| | | Extract¹ | SNAC | | | | |
|---|---|---|---|---|---|---|---|
| | | Dose | Dose | Dose | Dose | Dose | |
| Group | Group | Level | Level | Volume | Concentration² | Concentration³ | Number |
| Nos | Designation | (mg/kg) | (mg/kg) | (mL/kg) | (mg API/mL) | (mg SNAC/mL) | of Males |
| 1 | Excipient | 0 | 500 | 2 | 0 | 250 | 6 |
| | Control | | | | | | |
| 2 | Cannabis | 25 | 0 | 2 | 12.5 | 0 | 6 |
| | Control | | | | | | |
| 3 | Low Dose | 25 | 250 | 2 | 12.5 | 125 | 6 |
| 4 | Mid Dose | 25 | 500 | 2 | 12.5 | 250 | 6 |
| 5 | High Dose | 50 | 500 | 2 | 25 | 250 | 6 |

¹Extract contains 54% by weight (27% THC + 27% CBD) as the API (Active Pharmaceutical Ingredient)
²Dose of cannabis extract contains a mixture of THC:CBD in a ratio of 1:1 by weight
³SNAC dose is 10 times (THC + CBD) dose for groups 3 and 5 and 20 times for group 4.

Methods. Animals were dosed on Day 1 and a series of blood samples were collected over a period of 4 hours post dose for pharmacokinetic evaluation. Animals were euthanized following collection of their last blood sample.

Results. Following a single oral administration of cannabis extract containing THC/CBD in a 1:1 ratio combined with the absorption enhancing excipient (SNAC) at 25 mg extract/kg and 250 mg SNAC/kg (Group 3), 25 mg extract/kg and 500 mg SNAC/kg (Group 4), or 50 mg extract/kg and 500 mg SNAC/kg (Group 5), mean maximum concentration Cₘₐₓ ranged from 31.7 to 159.3 ng/mL for CBD and from 111.5 to 546.17 ng/mL for THC. The time to reach the mean maximum plasma concentration (Tₘₐₓ) ranged from 0.25 to 1 hour post dose for CBD and was reached at 1 hour post dose for the low and mid dose groups and at 2 hours post dose for the high dose group for THC. The AUC_{0-Tlast} ranged from 13.17 to 382.14 hr*ng/mL for CBD and from 170.64 to 1256.49 hr*ng/mL for THC.

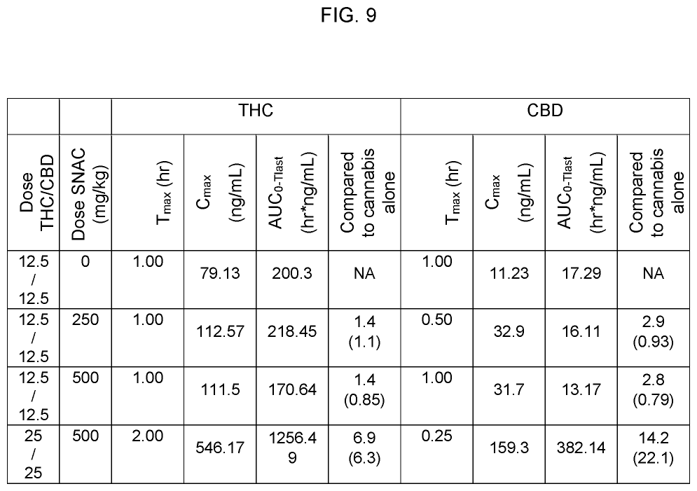

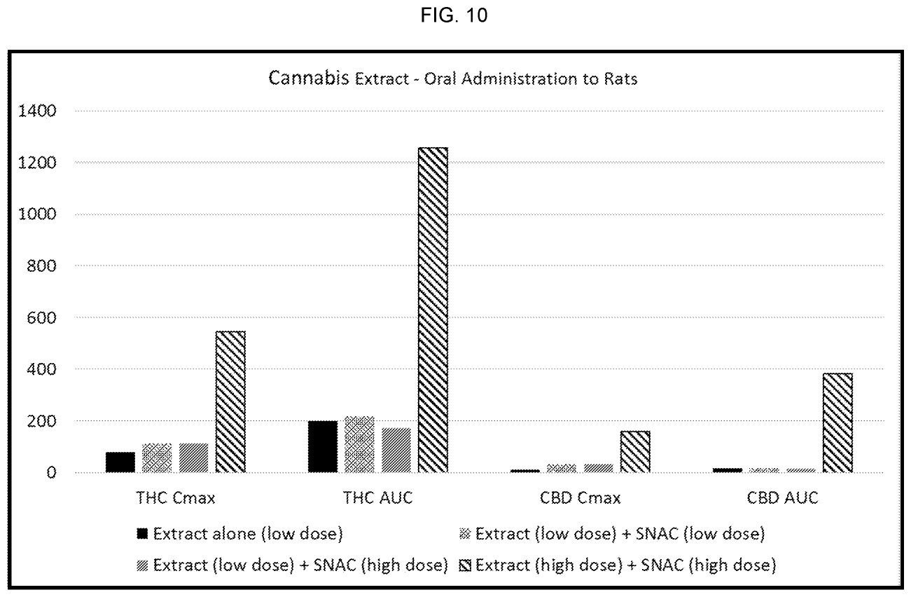

Over the dose range tested, Cₘₐₓ and AUC_{0-Tlast}, for THC was higher than for CBD. When administering the same cannabis extract (THC/CBD) dose (25 mg/kg total cannabinoid dose; 12.5 mg/kg THC/12.5 mg/kg CBD) with and without SNAC, for THC, a 1.4-fold Cₘₐₓ increase over cannabis alone was observed at SNAC doses of either 250 or 500 mg/kg. AUC was 1.1-fold greater in the 250 mg/kg SNAC group, but lower in the 500 mg/kg SNAC group, compared to the cannabis alone group. For CBD, 2.9-fold and 2.8-fold Cₘₐₓ increases over cannabis alone were observed at SNAC doses of either 250 or 500 mg/kg. AUC was lower in both groups, compared to the cannabis alone group. Increasing both the cannabis and SNAC doses 2-fold to 500 mg/kg SNAC and 50 mg/kg cannabis extract (25 mg/kg THC/25 mg/kg CBD), resulted in a 14.2-fold increase in the CBD Cₘₐₓ and a 6.9-fold increase in the THC Cₘₐₓ. AUC_{0-Tlast} for CBD and THC, were increased 22.1-fold and 6.3-fold, respectively (FIG. 9 and FIG. 10). Over the dose range tested, Cₘₐₓ and AUC_{0-Tlast}, for THC was higher than for CBD. When administering the same cannabis extract (THC/CBD) dose in the presence of SNAC (250 mg/kg or 500 mg/kg), both THC and CBD Cₘₐₓ were increased 1.4-fold and 2.8-fold, respectively, over the cannabis alone group. AUC_{0-Tlast} were comparable. This observation suggests that a cannabis to SNAC ratio of 10:1 facilitates an increase in Cₘₐₓ, but increasing the ratio to 20:1 provides no additional benefit. Increasing both the cannabis and SNAC doses by 2-fold resulted in THC and CBD Cₘₐₓ increases of 6.9-fold and 14.2-fold, respectively, over the cannabis alone group. AUC_{0-Tlast} for THC and CBD increased by 6.3-fold and 22.1-fold, respectively, over the cannabis alone group. This is a greater than expected increase based on the near linear dose response observed for oral cannabis (Information for Health Care Providers—Cannabis and the Cannabinoids; Health Canada February 2013). Overall, these data suggest that SNAC enhances cannabinoid absorption when administered to rats by oral gavage.

### Example 5

Onset and duration of action of orally administered cannabis/NAC composition. This study was designed to assess the utility of the acid form of SNAC, N-[8-(2-hydroxybenzoyl) amino] caprylic acid (NAC), in enabling a rapid-acting oral form of cannabis.

Study Participant. One study participant was recruited to ingest cannabis compositions and record the onset, duration, and intensity of cannabis-induced euphoria and/or dysphoria. The study participant took part in two separate tests: 1) use of a control substance, which included cannabis concentrate oil in an herbal extract blend dissolved in aqueous ethanol, and 2) use of a test substance, which included the cannabis concentrate oil in an herbal extract blend dissolved in aqueous ethanol, as well as NAC.

Formulations. The selected cannabis concentrate oil is commercially available in a capsule and the contents of the capsule were provided to the participant in an ethanol solution. One capsule contains 9 mg CBD, 7.7 mg THC, herbal extract blend (Magnolia bark, Ashwagandha, Astragalus), and stearic acid (from vegetable oil), and the stated potency per capsule is: CBD 9.0 mg, THCA 0.0 mg and THC 7.6 mg. The formulation was selected because it provides a noticeable effect on user-reported “euphoria”, and the CBD content should ameliorate dysphoric effects if Test 2 delivers a very high dose of cannabinoids.

Methods. For the Control experiment, the participant mixed the cannabis concentrate with 15 ml (one tablespoon) of aqueous ethanol, and immediately swallowed the mixture.

For the Test experiment, the participant mixed the cannabis concentrate with 15 ml pre-mixed solution of aqueous ethanol and 100 mg NAC, and immediately swallowed the dissolved mixture.

For both the Control experiment and the Test experiment, the participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-5. Table 5 shows descriptions of euphoria and dysphoria levels for each scale value.

TABLE 5
Scale Values for Reporting Euphoria and Dysphoria

| Scale Value | Description |
|---|---|
| 0 | No observed effect |
| 1 | Mild observed effect; possibly psychological |
| 2 | Definite but mild effect |
| 3 | Definite substantial effect |
| 4 | Strong effect |
| 5 | Intense effect |

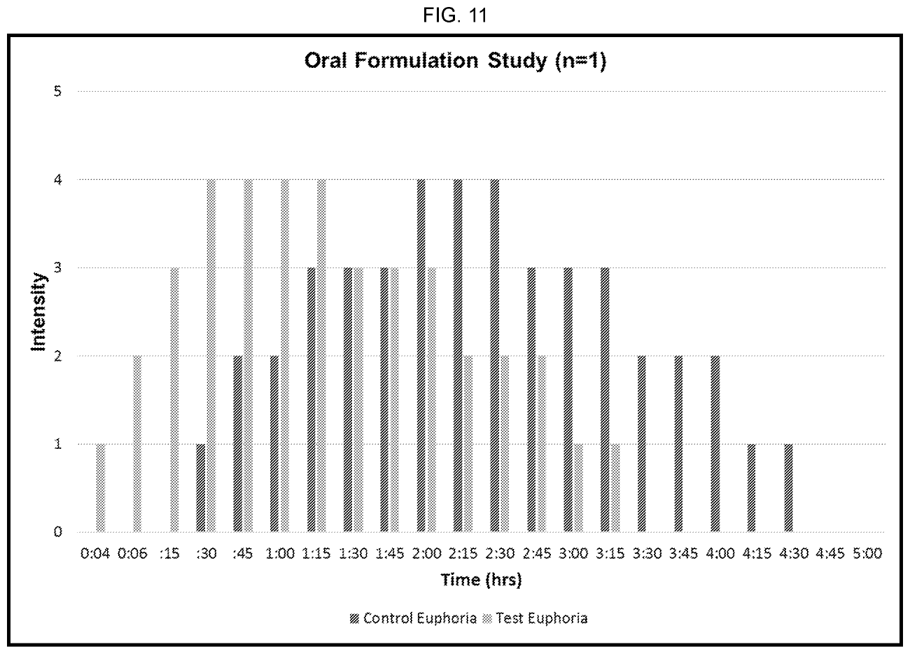

Results. The results shown below are scale values obtained for the participant in the control experiment (Table 6) and in the test experiment (Table 7). The values are plotted in FIG. 11.

TABLE 6
Control Experiment (n = 1)

| Actual | Time | Observed | Observed |
|---|---|---|---|
| Time | from Start | “Euphoria” | “Dysphoria” |
| 11:13 AM | 0:00 | (0-5) | (0-5) |
| 11:28 AM | :15 | 0 | 0 |
| 11:43 AM | :30 | 1 | 0 |
| 11:58 AM | :45 | 2 | 0 |
| 12:13 PM | 1:00 | 2 | 0 |
| 12:28 PM | 1:15 | 3 | 1 |
| 12:43 PM | 1:30 | 3 | 1 |
| 12:58 PM | 1:45 | 3 | 1 |
| 1:13 PM | 2:00 | 4 | 1 |
| 1:28 PM | 2:15 | 4 | 1 |
| 1:43 PM | 2:30 | 4 | 1 |
| 1:58 PM | 2:45 | 3 | 0 |
| 2:13 PM | 3:00 | 3 | 0 |
| 2:28 PM | 3:15 | 3 | 0 |
| 2:43 PM | 3:30 | 2 | 0 |
| 2:58 PM | 3:45 | 2 | 0 |
| 3:13 PM | 4:00 | 2 | 0 |
| 3:28 PM | 4:15 | 1 | 0 |
| 3:43 PM | 4:30 | 1 | 0 |
| 3:58 PM | 4:45 | 0 | 0 |
| 4:13 PM | 5:00 | 0 | 0 |

TABLE 7
Test Experiment (n = 1)

| Actual | Time | Observed | Observed |
|---|---|---|---|
| Time | from Start | “Euphoria” | “Dysphoria” |
| 11:20 AM | 0:00 | (0-5) | (0-5) |
| 11:24 AM | :04 | 1 | 0 |
| 11:26 AM | :06 | 2 | 0 |
| 11:35 AM | :15 | 3 | 1 |
| 11:50 AM | :30 | 4 | 0 |
| 12:05 PM | :45 | 4 | 0 |
| 12:20 PM | 1:00 | 4 | 0 |
| 12:35 PM | 1:15 | 4 | 0 |
| 12:50 PM | 1:30 | 3 | 0 |
| 1:05 PM | 1:45 | 3 | 0 |
| 1:20 PM | 2:00 | 3 | 0 |
| 1:35 PM | 2:15 | 2 | 0 |
| 1:50 PM | 2:30 | 2 | 0 |
| 2:05 PM | 2:45 | 2 | 0 |
| 2:20 PM | 3:00 | 1 | 0 |
| 2:35 PM | 3:15 | 1 | 0 |
| 2:50 PM | 3:30* | 0 | 0 |

*experiment ended

Onset: The participant reported euphoria within six minutes of ingesting the cannabis/NAC formulation (Test, Table 7 and FIG. 11). In contrast, the first time-point of euphoria reported by the participant after ingestion of the cannabis-only formulation (Control, Table 6 and FIG. 11) was forty-five minutes post-ingestion. By thirty minutes post-ingestion, the participant reported strong euphoria (scale value of 4) for the cannabis/NAC formulation. In contrast, thirty minutes after ingestion of the cannabis-only formulation, the participant only observed a mild effect that was possibly psychological (scale value of 1).

Intensity: The peak euphoria scale value after ingestion of both the cannabis-only (Control) and cannabis/NAC formulations (Test) was 4. However, peak intensity of euphoria was reached thirty minutes post-ingestion with the cannabis/NAC formulation (Test), whereas peak intensity of euphoria was reached two hours post-ingestion with the cannabis-only formulation (Control). Therefore, ingestion of the cannabis/NAC formulation led to a peak intensity of euphoria that occurred one hour and thirty minutes faster than when the cannabis-only formulation was ingested. The intensity of observed dysphoria was minimal for both the Test and Control, although the participant observed more mild dysphoria effects with the cannabis-only formulation (Control).

In summary, NAC, the acid form of SNAC, behaves similarly to SNAC when included in an oral dosage cannabinoid formulation, A cannabinoid/NAC formulation provides faster onset of action as compared to a cannabinoid-only formulation.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in an administration benefit when assessed in an animal model disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A composition comprising:
nabilone, and
sodium N-[8-(2-hydroxybenzoyl) amino] caprylate (SNAC),
wherein the SNAC and the nabilone are present at a ratio (w/w) of between 1:1 and 100:1.

2. An oral formulation of a composition of claim 1 comprising a therapeutically effective amount of the nabilone.

3. An oral formulation of claim 2 wherein the SNAC is at a concentration of 1-500 mg/mL.

4. A method of treating a symptom of a disease or disorder in a human subject, comprising administering an oral formulation of claim 2 to the subject, thereby treating the one or more symptoms of the disease or disorder in the human subject,
wherein the disease or disorder comprises acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

5. A composition comprising one or more synthetic cannabinoids or one or more salts thereof and one or more N-acylated fatty amino acids or salts thereof.

6. The composition of claim 5, wherein the one or more synthetic cannabinoids comprise Δ9-Tetrahydrocannabinol (THC) cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), or tetrahydrocannabivarinic acid (THCVA).

7. The composition of claim 5, wherein the one or more synthetic cannabinoids comprise a derivative and/or analog of a synthetic cannabinoid of claim 6 or a mixture thereof.

8. The composition of claim 5, wherein the one or more synthetic cannabinoids comprise 3-carbamoyl-2-pyridone or its derivatives and/or analogs; pyrimidine derivatives and/or analogs; carenadiol or its derivatives and/or analogs; cannabinoid carboxylic acids or their derivatives and/or analogs; pyrido[3,2-E][1,2,4]triazolo[4,3-C]pyrimidine or its derivatives and/or analogs; tetrahydro-pyrazolo[3,4-C] pyridine or its derivatives and/or analogs; bicyclo[3.1.1]heptan-2-one cannabinoid or its derivatives and/or analogs; resorcinol or its derivatives and/or analogs; dexanbinol compounds or their derivatives and/or analogs; cannabimimetic lipid amide compounds or their derivatives and/or analogs; nabilone or its derivatives and/or analogs; 2-oxoquinolone compounds or their derivatives and/or analogs disclosed; or 3,4-diaryl-4,5-dihydro-(h)-pyrazole-1-carboxamide or its derivatives and/or analogs.

9. The composition of claim 5, wherein the one or more N-acylated fatty amino acids or salts thereof comprise one or more of Compounds I-XXXV, or Formulas a-s.

10. The composition of claim 5, wherein the one or more N-acylated fatty amino acids or salts thereof comprise Sodium-N-salicyloyl-8-aminocaprylate, Monosodium 8-(N-salicyloylamino) octanoate, N-(salicyloyl)-8-aminooctanoic acid monosodium salt, monosodium N-{8-(2phenoxybenzoyl)amino}octanoate, or sodium 8-[(2-hydroxybenzoyl)amino]octanoate.

11. The composition of claim 5 wherein the one or more N-acylated fatty amino acids or salts thereof comprise 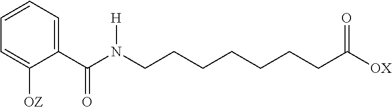
wherein X and Z are independently H, a monovalent cation, a divalent metal cation, or an organic cation.

12. The composition of claim 11, wherein the monovalent cation is sodium or potassium.

13. The composition of claim 11, wherein the metal cation is calcium or magnesium.

14. The composition of claim 11, wherein the organic cation is ammonium or tetramethylammonium.

15. The composition of claim 11, wherein X is H.

16. The composition of claim 11, wherein X is a monovalent cation comprising sodium or potassium.

17. The composition of claim 11, wherein X is a divalent metal cation comprising calcium or magnesium.

18. The composition of claim 11, wherein X is an organic cation comprising ammonium or tetramethylammonium.

19. The composition of claim 11, wherein Z is H.

20. The composition of claim 11, wherein Z is a monovalent cation comprising sodium or potassium.

21. The composition of claim 11, wherein Z is a divalent cation comprising calcium or magnesium.

22. The composition of claim 11, wherein X is H and Z is H.

23. The composition of claim 11, wherein X is H and Z is sodium.

24. The composition of claim 11, wherein X is sodium and Z is sodium.

25. The composition of claim 5, wherein the one or more N-acylated fatty amino acids or salts thereof provide an administration benefit.

26. The composition of claim 25, wherein the administration benefit is a dose-dependent administration benefit.

27. The composition of claim 26, wherein the dose-dependent administration benefit is at a dose of 100-200 mg.

28. The composition of claim 26, wherein the dose-dependent administration benefit is at a dose concentration of 100 mg/mL to 300 mg/mL N-acylated fatty amino acid or salt thereof.

29. The composition of claim 26, wherein the dose-dependent administration benefit is at a dose concentration of 1-500 mg/mL N-acylated fatty amino acid or salt thereof.

30. The composition of claim 26, wherein the dose-dependent administration benefit is at a dose concentration of 250 mg/mL N-acylated fatty amino acid or salt thereof.

31. The composition of claim 26, wherein the dose-dependent administration benefit of the N-acylated fatty amino acid or salt thereof is at a dose of one to one hundred times the dose of the one or more synthetic cannabinoids.

32. An oral formulation, wherein the oral formulation comprises the composition of claim 25 and wherein the administration benefit includes one or more of increased absorption of a measured component of the synthetic cannabinoid, increased bioavailability of a measured component of the synthetic cannabinoid, faster onset of action of a measured component of the synthetic cannabinoid, higher peak concentrations of a measured component of the synthetic cannabinoid, faster time to peak concentrations of a measured component of the synthetic cannabinoid, increased subjective therapeutic efficacy, increased objective therapeutic efficacy, improved taste, and improved mouthfeel as compared to a control composition without the N-acylated fatty amino acid or salt thereof.

33. The composition of claim 5, wherein the composition is a pharmaceutical composition.

34. The composition of claim 5, wherein the composition is a nutritional supplement and further comprises at least one vitamin and/or mineral.

35. The composition of claim 5, wherein the composition further comprises a surfactant, detergent, azone, pyrrolidone, glycol or bile salt.

36. The composition of claim 5, wherein the composition comprises a therapeutically effective amount of the one or more synthetic cannabinoids.

37. The composition of claim 36 wherein the therapeutically effective amount of the one or more synthetic cannabinoids treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

38. The composition of claim 5, wherein the composition further comprises one or more vitamins or one or more minerals.

39. The composition of claim 5, wherein the composition further comprises one or more vitamins and one or more minerals.

40. The composition of claim 38, wherein the one or more vitamins comprise Vitamin A, Vitamin B1, Vitamin B6, Vitamin B12, Vitamin C, Vitamin D, Vitamin E, or Vitamin K.

41. The composition of claim 38, wherein the one or more minerals comprise calcium, chromium, iodine, iron, magnesium, selenium, or zinc.

42. An oral formulation, wherein the oral formulation comprises the composition of claim 5.

43. The oral formulation of claim 42, wherein the oral formulation is swallowable or chewable.

44. The oral formulation of claim 42, wherein the oral formulation is liquid or solid.

45. The oral formulation of claim 42, wherein the oral formulation is a solution, suspension, or spray.

46. The oral formulation of claim 42, wherein the oral formulation is a tablet, capsule or sachet.

47. The oral formulation of claim 42, wherein the oral formulation is flavored.

48. A method of treating a subject in need thereof including administering a therapeutically effective amount of a composition of claim 5 or a formulation of claim 42 to the subject thereby treating the subject in need thereof.

49. A method of claim 48 wherein the therapeutically effective amount provides an effective amount, a prophylactic treatment, and/or a therapeutic treatment.

50. A method of reducing or eliminating one or more symptoms of a disease or disorder in a human subject,
wherein said method comprises delivering a therapeutically effective amount of a composition of claim 5 or oral formulation of claim 42 to the subject, thereby reducing or eliminating one or more symptoms of the disease or disorder, and
wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Graves' disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

51. The method of claim 50, wherein the therapeutically effective amount of the composition comprises (i) an amount of the N-acylated fatty amino acid or salt thereof and (ii) an amount of the one or more synthetic cannabinoids that are at a ratio (w/w) of between 1:1 and 100:1.

52. A method of preparing a synthetic cannabinoid oral formulation having a faster onset of action, wherein the method comprises adding an absorption enhancer to a synthetic cannabinoid, and wherein the synthetic cannabinoid oral formulation has a faster onset of action than a synthetic cannabinoid oral formulation without an absorption enhancer.

53. The method of claim 52, wherein the absorption enhancer comprises an N-acylated fatty amino acid or a salt thereof.

54. The method of claim 53, wherein the N-acylated fatty amino acid comprises N-[8-(2-hydroxybenzoyl) amino] caprylate.

55. The method of claim 52, wherein the absorption enhancer and the synthetic cannabinoid are present at a ratio (w/w) of between 1:1 and 100:1.

56. The method of claim 52, wherein the absorption enhancer and the synthetic cannabinoid are present at a ratio (w/w) of between 1:1 and 20:1.
